SEQUENCE LISTING (34 sequences)

SEQ ID NO: 1 - protein, 24 aa, Homo sapiens
  region B of PRK2
REPRILSEEEQEMFRDFDYIADWC

SEQ ID NO: 2 - protein, 24 aa, Artificial Sequence
  Description of Artificial Sequencesynthetic peptide sequence that interacts with human PRK2 region B
REPRILSEEEQEMARDFDYIADWC

SEQ ID NO: 3 - protein, 24 aa, Artificial Sequence
  Description of Artificial Sequencesynthetic peptide sequence that interacts with human PRK2 region B
REPRILSEEEQEMFGDFDYIADWC

SEQ ID NO: 4 - protein, 53 aa, Homo sapiens
  region A of PRK2
EDVKKHPFFRLIDWSALMDKKVKPPFIPTIRGREDVSNFDDEFTSEAPILTPP

SEQ ID NO: 5 - protein, 23 aa, Homo sapiens
  region of PKC zeta
DEDAIKRIDQSEFEGFEYINPLL

SEQ ID NO: 6 - protein, 6 aa, Homo sapiens
  portion of region B of PRK2
FRDFDY

SEQ ID NO: 7 - protein, 23 aa, Homo sapiens
  region of PKC zeta
DEDAIKRIDQSEFEGFEYINPLL

SEQ ID NO: 8 - protein, 11 aa, Homo sapiens
  region of PKB alpha
PHFPQFSYSAS

SEQ ID NO: 9 - protein, 9 aa, Homo sapiens
  region of PRK1
TFCGTPEFL

SEQ ID NO: 10 - protein, 6 aa, Homo sapiens
  region of PKC zeta
FEGFEY

SEQ ID NO: 11 - protein, 13 aa, Homo sapiens
  region of PDK1
RQRYQSHPDAAVQ

SEQ ID NO: 12 - DNA, 28 bp, Artificial Sequence
  Description of Artificial Sequencepcr primer
cgggatccgaggatgtaaaaaagcaccc

SEQ ID NO: 13 - protein, 7 aa, Artificial Sequence
  Description of Artificial Sequencehuman PKB alphasubstrate peptide
RPRTAAF

SEQ ID NO: 14 - protein, 77 aa, Homo sapiens
  region of PRK2
EDVKKHPFFRLIDWSALMDKKVKPPFIPTIRGREDVSNFDDEFTSEAPILTPPREPRILSEEEQEMFRDFDYIADWC

SEQ ID NO: 15 - protein, 77 aa, Homo sapiens
  region of PRK1
EDVKKQPFFRTLGWEALLARRLPPPFVPTLSGRTDVSNFDEEFTGEAPTLSPPRDARPLTAAEQAAFLDFDFVAGGC

SEQ ID NO: 16 - protein, 80 aa, Homo sapiens
  region of PKB alpha
KEIMQHRFFAGIVWQHVYEKKLSPPFKPQVTSETDTRYFDEEFTAQMITITPPDQDDSMECVDSERRPHFPQFSYSASTA

SEQ ID NO: 17 - protein, 75 aa, Homo sapiens
  region of P70S6k
GEVQAHPFFRHINWEELLARKVEPPFKPLLQSEEDVSQFDSKFTRQTPVDSPDDSTLSESANQVFLGFTYVAPSV

SEQ ID NO: 18 - protein, 82 aa, Homo sapiens
  region of SGK
MEIKSHVFFSLINWDDLINKKITPPFNPNVSGPNELRHFDPEFTEEPVPNSIGKSPDSVLVTASVKEAAEAFLGFSYAPPTD

SEQ ID NO: 19 - protein, 76 aa, Homo sapiens
  region of PKC zeta
SDIKSHAFFRSIDWDLLEKKQALPPFQPQITDDYGLDNFDTQFTSEPVQLTPDDEDAIKRIDQSEFEGFEYINPLL

SEQ ID NO: 20 - protein, 75 aa, Homo sapiens
  region of PKC alpha
RDVREHAFFRRIDWEKLENREIQPPFKPKVCGKGAENFDKFFTRGQPVLTPPDQLVIANIDQSDFEGFSYVNPQF

SEQ ID NO: 21 - protein, 62 aa, Homo sapiens
  region of PKA beta
SDIKTHKWFATTDWIAIYQRKVEAPFIPKFGRSGDTSNFDDYEEEDIRVSITECAKEKFGEF

SEQ ID NO: 22 - protein, 6 aa, Homo sapiens
  PKB consensus sequence
  residues 2 and 3 are variable amino acids
FXXFSF

SEQ ID NO: 23 - protein, 8 aa, Homo sapiens
  PKB consensus sequence
  residue 6 is a variable amino acid
TFCGTXEL

SEQ ID NO: 24 - protein, 6 aa, Homo sapiens
  PKB consensus sequence
  residues 2 and 3 are variable amino acids
  residue 5 is serine or threonine
  residue 6 is phenylalanine or tyrosine
FXXFXX

SEQ ID NO: 25 - protein, 6 aa, Artificial Sequence
  Description of Artificial Sequencesynthetic sequence that interacts with PKB
  residues 2 and 3 are variable amino acids
  residues 1, 4 and 6 are phenylalanine or tyrosine
  residue 5 is a negatively charged amino acid
XXXXXX

SEQ ID NO: 26 - protein, 6 aa, Artificial Sequence
  Description of Artificial Sequencesynthetic sequence that interacts with PKB
  residues 2 and 3 are variable amino acids
  residues 1, 4 and 6 are phenylalanine or tyrosine
  residue 5 is aspartic acid or glutamic acid
XXXXXX

SEQ ID NO: 27 - protein, 6 aa, Artificial Sequence
  Description of Artificial Sequencesynthetic sequence that interacts with PKB
  residues 2 and 3 are variable amino acids
  residues 1, 4 and 6 are phenylalanine or tyrosine
  residue 5 is phospho serine or phospho threonine
XXXXXX

SEQ ID NO: 28 - protein, 8 aa, Homo sapiens
  PKB consensus sequence
  residue 1 is threonine or serine
  residue 6 is a variable amino acid
XFCGTXEL

SEQ ID NO: 29 - protein, 6 aa, Homo sapiens
  C terminal region of PKC zeta
FEGFEY

SEQ ID NO: 30 - protein, 6 aa, Homo sapiens
  PKB consensus sequence
  residues 1, 4 and 6 are phenylalanine or tyrosine
  residues 2 and 3 are variable amino acids
  rsidue 5 is serine or threonine
XXXXXX

SEQ ID NO: 31 - protein, 556 aa, Homo sapiens
  PDK1 sequence
MARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQFVENNLILKMGPVDKRKGLFARRRQLLLTEGPHLYYVDPVNKVLKGEIPWSQELRPEAKNFKTFFVHTPNRTYYLMDPSGNAHKWCRKIQEVWRQRYQSHPDAAVQ

SEQ ID NO: 32 - protein, 984 aa, Homo sapiens
  PRK2 sequence
MASNPERGEILLTELQGDSRSLPFSENVSAVQKLDFSDTMVQQKLDDIKDRIKREIRKELKIKEGAENLRKVTTDKKSLAYVDNILKKSNKKLEELHHKLQELNAHIVVSDPEDITDCPRTPDTPNNDPRCSTSNNRLKALQKQLDIELKVKQGAENMIQMYSNGSSKDRKLHGTAQQLLQDSKTKIEVIRMQILQAVQTNELAFDNAKPVISPLELRMEELRHHFRIEFAVAEGAKNVMKLLGSGKVTDRKALSEAQARFNESSQKLDLLKYSLEQRLNEVPKNHPKSRIIIEELSLVAASPTLSPRQSMISTQNQYSTLSKPAALTGTLEVRLMGCQDILENVPGRSKATSVALPGWSPSETRSSFMSRTSKSKSGSSRNLLKTDDLSNDVCAVLKLDNTVVGQTSWKPISNQSWDQKFTLELDRSRELEISVYWRDWRSLCAVKFLRLEDFLDNQRHGMCLYLEPQGTLFAEVTFFNPVIERRPKLQRQKKIFSKQQGKTFLRAPQMNINIATWGRLVRRAIPTVNHSGTFSPQAPVPTTVPVVDVRIPQLAPPASDSTVTKLDFDLEPEPPPAPPRASSLGEIDESSELRVLDIPGQDSETVFDIQNDRNSILPKSQSEYKPDTPQSGLEYSGIQELEDRRSQQRFQFNLQDFRCCAVLGRGHFGKVLLAEYKNTNEMFAIKALKKGDIVARDEVDSLMCEKRIFETVNSVRHPFLVNLFACFQTKEHVCFVMEYAAGGDLMMHIHTDVFSEPRAVFYAACVVLGLQYLHEHKIVYRDLKLDNLLLDTEGFVKIADFGLCKEGMGYGDRTSTFCGTPEFLAPEVLTETSYTRAVDWWGLGVLIYEMLVGESPFPGDDEEEVFDSIVNDEVRYPRFLSTEAISIMRRLLRRNPERRLGASEKDAEDVKKHPFFRLIDWSALMDKKVKPPFIPTIRGREDVSNFDDEFTSEAPILTPPREPRILSEEEQEMFRDFDYIADWC

SEQ ID NO: 33 - protein, 942 aa, Homo sapiens
  PRK1 sequence
MASDAVQSEPRSWSLLEQLGLAGADLAAPGVQQQLELERERLRREIRKELKLKEGAENLRRATTDLGRSLGPVELLLRGSSRRLDLLHQQLQELHAHVVLPDPAATHDGPQSPGAGGPTCSATNLSRVAGLEKQLAIELKVKQGAENMIQTYSNGSTKDRKLLLTAQQMLQDSKTKIDIIRMQLRRALQADQLENQAAPDDTQGSPDLGAVELRIEELRHHFRVEHAVAEGAKNVLRLLSAAKAPDRKAVSEAQEKLTESNQKLGLLREALERRLGELPADHPKGRLLREELAAASSAAFSTRLAGPFPATHYSTLCKPAPLTGTLEVRVVGCRDLPETIPWNPTPSMGGPGTPDSRPPFLSRPARGLYSRSGSLSGRSSLKAEAENTSEVSTVLKLDNTVVGQTSWKPCGPNAWDQSFTLELERARELELAVFWRDQRGLCALKFLKLEDFLDNERHEVQLDMEPQGCLVAEVTFRNPVIERIPRLRRQKKIFSKQQGKAFQRARQMNIDVATWVRLLRRLIPNATGTGTFSPGASPGSEARTTGDISVEKLNLGTDSDSSPQKSSRDPPSSPSSLSSPIQESTAPELPSETQETPGPALCSPLRKSPLTLEDFKFLAVLGRGHFGKVLLSEFRPSGELFAIKALKKGDIVARDEVESLMCEKRILAAVTSAGHPFLVNLFGCFQTPEHVCFVMEYSAGGDLMLHIHSDVFSEPRAIFYSACVVLGLQFLHEHKIVYRDLKLDNLLLDTEGYVKIADFGLCKEGMGYGDRTSTFCGTPEFLAPEVLTDTSYTRAVDWWGLGVLLYEMLVGESPFPGDDEEEVFDSIVNDEVRYPRFLSAEAIGIMRRLLRRNPERRLGSSERDAEDVKKQPFFRTLGWEALLARRLPPPFVPTLSGRTDVSNFDEEFTGEAPTLSPPRDARPLTAAEQAAFLDFDFVAGGC

SEQ ID NO: 34 - protein, 592 aa, Homo sapiens [sequence omitted]

(19) United States
(12) Patent Application Publication
(10) Pub. No.: US 2007/0196883 A1
(43) Pub. Date: Aug. 23, 2007

(54) ENZYME

(75) Inventors: Dario Alessi, Dundee (GB); Anudharan Balendran, Dundee (GB); Maria Deak, Dundee (GB); Richard Currie, Dundee (GB); Peter Downes, Wormit (GB); Antonio Casamayor, New Haven, CT (US)

Correspondence Address: ROGALSKY & WEYAND, LLP, P.O. BOX 1927, WILLIAMSVILLE, NY, 14231-1927, US

(73) Assignee: UNIVERSITY OF DUNDEE, Dundee (GB)

(21) Appl. No.: 11/671,265

(22) Filed: Feb. 5, 2007

Related U.S. Application Data
(63) Continuation of application No. 09/937,009, filed on Apr. 2, 2002, now abandoned.

(30) Foreign Application Priority Data
Mar. 19, 1999 (GB) 9906245.7

(51) Int. Cl. C12Q 1/48; C07H 21/04; C12P 21/06; C12N 9/12

(52) U.S. Cl. 435/15; 435/69.1; 435/320.1; 435/194; 435/325; 536/23.2

(57) ABSTRACT

The present invention relates to methods of altering the substrate specificity of PDK1, methods of identifying compounds which modulate the activity of PDK1 and methods of using PDK1 having altered substrate specificity.

This application is a continuation of U.S. patent application Ser. No. 09/937,009, which is a National Stage of PCT/GB00/01004, filed Mar. 17, 2000, which is hereby incorporated by reference herein.

The present invention relates to enzymes, in particular to phosphoinositide-dependent protein kinase 1 (PDK1).

Protein kinase B (PKB) is activated by a phosphoinositide 3-kinase dependent pathway when cells are stimulated with insulin or growth factors. The mechanism involves the phosphorylation of Thr308 located in the kinase domain of PKB and Ser 473 which is situated in a hydrophobic motif -Phe-Xaa-Xaa-Phe-Ser-Phe- (SEQ ID NO:22) located close to its C-terminus. Thr308 is phosphorylated by PDK1 in vitro, but the identity of the kinase that phosphorylates Ser473 (provisionally termed PDK2) is not known.

The first steps in insulin signal transduction lead to the activation of phosphoinositide (PI) 3-kinase and the formation of PtdIns(3,4,5)P₃ at the plasma membrane [1-3], which can then be converted to PtdIns(3,4)P_{2 [}4] or to PtdIns(4,5)P_{2 [}5,6] by specific phosphatases. The inhibition of PI 3-kinase suppresses nearly all the metabolic actions of insulin whereas expression of constitutively active forms of these enzymes mimics these responses in the absence of insulin. These findings demonstrate that PtdIns(3,4,5)P₃ (and/or PtdIns(3,4)P₂) are key second messengers for insulin signalling [reviewed in 1-3].

Protein kinase B (PKB) is activated very rapidly in response to insulin, its activation is prevented by inhibitors of PI 3-kinase [7], and further studies have revealed that PKB is likely to mediate many of the effects of insulin in cells including stimulation of glycogen synthesis in skeletal muscle [8], the stimulation of glycolysis in cardiac muscle [9] and some of the effects that insulin has on gene transcription [10,11] and translation [12].

PKB contains an N-terminal pleckstrin-homology (PH) domain followed by a kinase catalytic domain and then a C-terminal tail. The catalytic domain of PKB belongs to the AGC subfamily of protein kinases and was originally identified by its similarity to that of protein kinase C (PKC) isoforms and protein kinase A (PKA) [13]. Recent work has led to a greater understanding of the mechanism by which PKB is activated. PKB (through its PH domain) interacts with PtdIns(3,4,5)P₃ the product of PI 3-kinase activation. This results in the translocation of PKB to the plasma membrane and a conformational change that allows it to be phosphorylated, but does not directly stimulate PKB activity [2, 3 and 13]. Instead, PKB is activated at the membrane by phosphorylation of two residues, namely Thr308 and Ser473. Both of these residues need to be phosphorylated for maximal activation of PKB and their phosphorylation in vivo is prevented by inhibitors of PtdIns 3-kinase [14]. Thr 308 is located between subdomains VII and VIII of the kinase domain, a region in which many kinases activated by phosphorylation are phosphorylated. Ser473 is located C-terminal to the catalytic domain, in a region that nevertheless displays high homology between different AGC family members. Interestingly, other members of the AGC subfamily of protein kinases, including p70 S6K [15] and PKC isoforms [16] also possess residues lying in equivalent sequences to Thr308 and Ser473 of PKB and phosphorylation of these residues is necessary for activation of these kinases in vivo. The residues equivalent to Thr308 lie in a Thr-Phe-Cys-Gly-Thr-Xaa-Glu-Leu (SEQ ID NO:23) consensus motif (where the underlined Thr corresponds to Thr308 and Xaa is a variable residue). The residues surrounding Ser473 lie in a Phe-Xaa-Xaa-Phe-Ser/Thr-Phe/Tyr (SEQ ID NO:24) consensus motif. We [17,18] and others [19,20] have identified a protein kinase termed 3-phosphoinositide dependent protein kinase-1 (PDK1) which, in the presence of PtdIns(3,4,5)P₃ or PtdIns(3,4)P₂, phosphorylates PKB at Thr308. Subsequent work has also shown that PDK1 phosphorylates the equivalent residues on PKC isoforms [21-23], p70 S6 kinase [24,25] and PKA [26]. The finding that PDK1 does not phosphorylate PKB significantly at Ser473, in vitro or in co-transfection experiments, suggested that a distinct protein kinase which provisionally has been termed PDK2 may catalyse this reaction [17]. Here we present findings that indicate that, surprisingly, this may not be the case. In particular we demonstrate that PDK1 can be converted to a form that phosphorylates both Ser473 and Thr308 by interaction with a small peptide, for example a peptide corresponding to the C-terminal region of protein kinase C-related protein kinase-2 (PRK2). These observations suggest that PDK1 and PDK2 may be the same enzyme and that the specificity of PDK1 towards Thr308 and Ser473 may be regulated through its interaction with other cellular components.

A first aspect of the invention provides a method of altering the substrate specificity of phosphoinositide-dependent protein kinase 1 (PDK1) wherein the said PDK1 is exposed to a polypeptide which comprises the amino acid sequence Phe/Tyr-Xaa-Xaa-Phe/Tyr-Zaa-Phe/Tyr (SEQ ID NO:25) wherein Zaa represents a negatively charged amino acid residue.

The negatively charged amino acid residue Zaa may be, for example, an aspartate, glutamate, phosphorylated serine (phosphoserine), phosphorylated threonine (phosphothreonine) or phosphorylated tyrosine (phosphotyrosine) residue, or a negatively charged non-naturally occuring residue. It is preferred that Zaa is an aspartate, glutamate, phosphoserine or phosphothreonine residue, still more preferably an aspartate or glutamate residue. It is preferred that the first residue in the sequence corresponding to the above consensus sequence is a phenylalanine residue. Phenylalanine is found in this position in naturally occuring polypeptides in which the consensus sequence has been identified. It may also be preferred that the residue preceding the Zaa residue is a phenylalanine residue. Phenylalanine and tyrosine are both (separately) found in this position in naturally occuring polypeptides in which the consensus sequence has been identified.

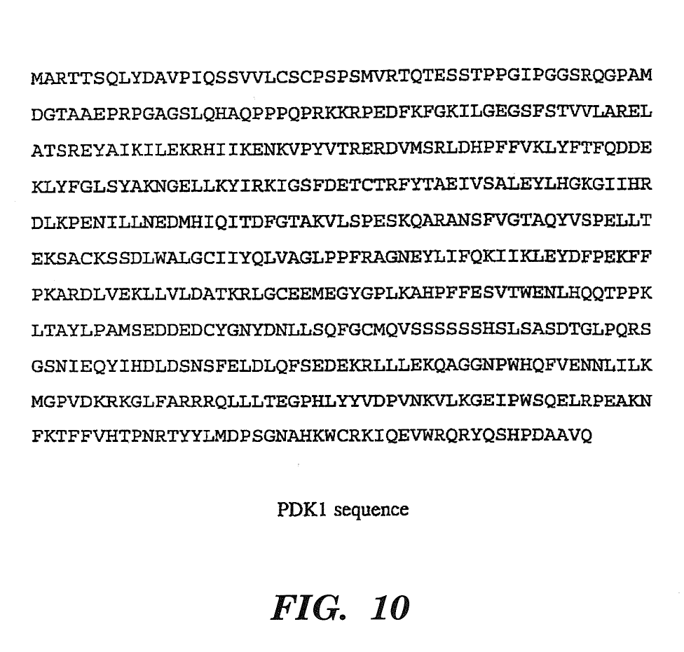

It will be appreciated that the term PDK1 as used herein includes a polypeptide (a PDK1 polypeptide) comprising the amino acid sequence shown in FIG. 10 or identified as PDK1 in Alessi D. R et al (1997) Characterisation of a 3-phosphoinositide-dependent protein kinase which phosphorylates and activates protein kinase B. Curr. Biol. 7: 261-269, Alessi D. R et al (1997) 3-phosphoinositide-dependent protein kinase-1 (PDK1): structural and functional homology with the Drosophila DSTPK61 kinase. Curr. Biol. 7: 776-789, Stokoe D et al (1997) Dual role of phosphatidyinositiol-3,4,5-trisphosphate in the activation of protein kinase B. Science 277: 567-570 or Stephens L et al (1998) Protein kinase B kinases that mediate phosphatidylinositol 3,4,5-trisphosphate-dependent activation of protein kinase B. Science 279: 710-714, or a variant, fragment, fusion or derivative thereof, or a fusion of a said variant or fragment or derivative, for example as described in WO98/41638. It is preferred that the said PDK1 polypeptide is a protein kinase. It is preferred that the said PDK1 polypeptide is a protein kinase that is capable of phosphorylating a threonine residue that lies in a Thr-Phe-Cys-Gly-Thr-Xaa-Glu-Leu (SEQ ID NO:23) consensus motif (where the underlined Thr corresponds to the threonine that is phosphorylated by PDK1 and Xaa is a variable residue), and preferably that is capable of phosphorylating PKB, for example PKBα, at residue Thr308. The rate at which the said PDK1 polypeptide is capable of phosphorylating a threonine residue as described above may be increased in the presence of PtdIns(3,4,5)P₃ or PtdIns(3,4)P₂. The said polypeptide may be capable of phosphorylating the equivalent residues to Thr308 of PKBα on PKC isoforms [21-23], p70 S6 kinase [24,25], SGK (sequence given in Webster, M. K., Goya, Ge, Y., Malyar, A. C. and Firestone, G. L. (1993) Mol. Cell. Biol. 13, 1031-2040; equivalent residues identified in US application no 112217 filed on 14 Dec. 1998) and PKA [26]. It may further be preferred that the substrate specificity and/or other characteristics of the said polypeptide in vitro may be substantially as reported in Alessi D. R et al (1997) Curr. Biol. 7: 261-269, Alessi D. R et al (1997) Curr. Biol. 7: 776-789, Stokoe D et al (1997) Science 277: 567-570 or Stephens L et al (1998) Science 279: 710-714.

It is particularly preferred, although not essential, that the variant or fragment or derivative or fusion of the PDK1, or the fusion of the variant or fragment or derivative has at least 30% of the enzyme activity of full-length human PDK1 with respect to the phosphorylation of full-length human PKBα on residue Thr308 in either the presence or absence of PtdIns(3,4,5)P₃ or PtdIns(3,4)P₂. It is more preferred if the variant or fragment or derivative or fusion of the said protein kinase, or the fusion of the variant or fragment or derivative has at least 50%, preferably at least 70% and more preferably at least 90% of the enzyme activity of PDK1 with respect to the phosphorylation of PKBα. However, it will be appreciated that variants or fusions or derivatives or fragments which are devoid of enzymatic activity may nevertheless be useful, for example by interacting with another polypeptide. Thus, variants or fusions or derivatives or fragments which are devoid of enzymatic activity may be useful in a binding assay, which may be used, for example, in a method of the invention in which an interaction of PDK1 (as defined above) with a polypeptide comprising the amino acid sequence motif Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27) is measured.

By “variants” of a polypeptide we include insertions, deletions and substitutions, either conservative or non-conservative. In particular we include variants of the polypeptide where such changes do not substantially alter the activity of the said polypeptide, for example the protein kinase activity of PDK1, as described above.

By “conservative substitutions” is intended combinations such as Gly, Ala; Val, Ile, Leu; Asp, Glu; Asn, Gln; Ser, Thr; Lys, Arg; and Phe, Tyr.

It is particularly preferred if the PDK1 variant has an amino acid sequence which has at least 65% identity with the amino acid sequence of PDK1 shown in FIG. 10, more preferably at least 70%, 71%, 72%, 73% or 74%, still more preferably at least 75%, yet still more preferably at least 80%, in further preference at least 85%, in still further preference at least 90% and most preferably at least 95% or 97% identity with the amino acid sequence defined above.

It is still further preferred if the PDK1 variant has an amino acid sequence which has at least 65% identity with the amino acid sequence of the catalytic domain of PDK1 shown in FIG. 10, more preferably at least 70%, 71%, 72%, 73% or 74%, still more preferably at least 75%, yet still more preferably at least 80%, in further preference at least 83 or 85%, in still further preference at least 90% and most preferably at least 95% or 97% identity with the amino acid sequence defined above. It will be appreciated that the catalytic domain of a protein kinase-related polypeptide may be readily identified by a person skilled in the art, for example using sequence comparisons as described below.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al (1994) Nucl Acid Res 22, 4673-4680). The parameters used may be as follows:

Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05. Scoring matrix: BLOSUM.

It is preferred that the PDK1 is a polypeptide which consists of the amino acid sequence of the protein kinase PDK1 as shown in FIG. 10 or naturally occurring allelic variants thereof. The PDK1 may also be a polypeptide with the amino acid sequence of residues 51 to 404 of full-length human PDK1; this may comprise the protein kinase domain of PDK1, as described in Example 1. The PDK1 may also be a Myc epitope-tagged PDK1 or His-tagged PDK1, as described in Example 1.

It is preferred that the PDK1 is a polypeptide that is capable of binding to a polypeptide comprising the amino acid sequence motif Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27).

The capability of the said PDK1 polypeptide with regard to binding a polypeptide comprising the amino acid sequence motif Phe/Tyr-Xaa-Xaa-Phe/Tyr-Zaa-Phe/Tyr (SEQ ID NO:25) may be measured by any method of detecting/measuring a protein/protein interaction, as discussed further below. Suitable methods include methods analagous to those discussed above and described in Example 1, for example yeast two-hybrid interactions, co-purification, ELISA or co-immunoprecipitation methods. Thus, the said PDK1 may be considered capable of binding a polypeptide comprising the amino acid sequence motif Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27) (the interacting polypeptide) if an interaction may be detected between the said PDK1 polypeptide and the said interacting polypeptide by ELISA, co-immunoprecipitation methods or by a yeast two-hybrid interaction or copurification method, for example as described in Example 1.

It is preferred that the interacting polypeptide comprising the amino acid sequence motif Phe/Tyr-Xaa-Xaa-Phe/Tyr-Zaa-Phe/Tyr (SEQ ID NO:25) is a polypeptide that is capable of binding PDK1 and changing its activity towards Ser473 of PKBα in substantially the same way as a polypeptide with the amino acid sequence REPRILSEEEQEMFRDFDYIADWC (SEQ ID NO:1), as described in Example 1.

The three-letter amino acid code of the IUPAC-IUB Biochemical Nomenclature Commission is used herein, with the exception of the symbol Zaa, defined above. In particular, Xaa represents any amino acid. It is preferred that Xaa and Zaa represent a naturally occuring amino acid. It is preferred that at least the amino acids corresponding to the consensus sequence are L-amino acids.

It is preferred that after the said exposure the PDK1 is capable of phosphorylating the underlined residue in a polypeptide with an amino acid sequence corresponding to the consensus sequence Phe-Xaa-Xaa-Phe-Ser/Thr-Phe/Tyr (SEQ ID NO:24). It is further preferred that after said exposure the PDK1 is capable of phosphorylating residue Ser473 of PKBα. It is still further preferred that after said exposure the protein kinase is also capable of phosphorylating the underlined residue in a polypeptide with an amino acid sequence corresponding to the consensus sequence Thr/Ser-Phe-Cys-Gly-Thr-Xaa-Glu-Leu (SEQ ID NO:28). It is still further preferred that after said exposure the protein kinase is capable of phosphorylating PKBα on Thr308 and Ser473.

Either said capability of PDK1 may be altered, preferably increased, in the presence of PtdIns(3,4,5)P₃ (PIP₃) or PtdIns(3,4,)P₂ dependent manner. The requirement for PtdIns(3,4,5)P₃ or PtdIns(3,4,)P₂ may be extremely specific, because only the D-enantiomers of PtdIns(3,4,5)P₃ appear to be effective, and many other PtdIns phospholipids including PtdIns(4,5)P₂ appear to be ineffective, as discussed in Example 1.

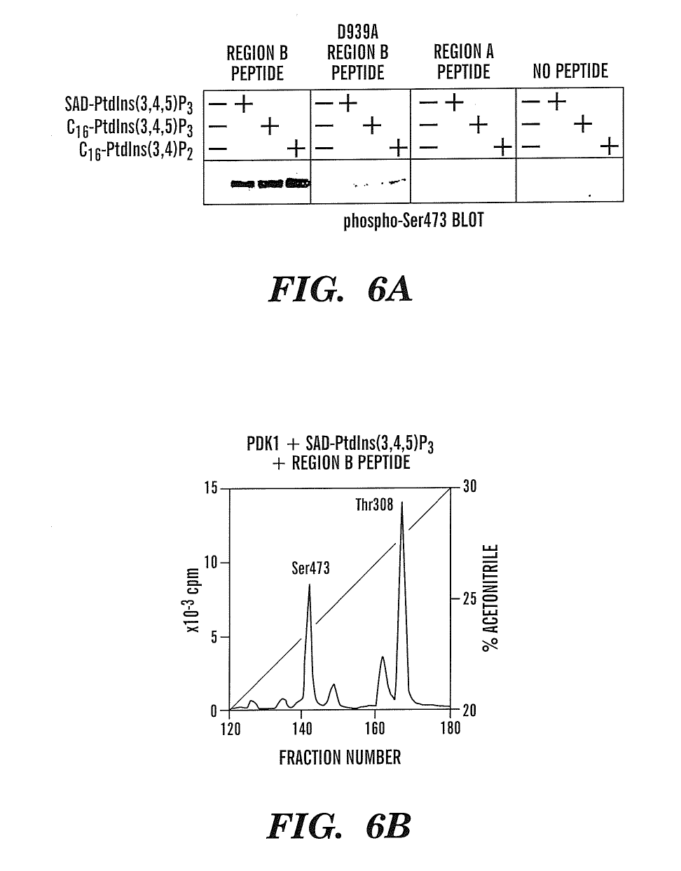

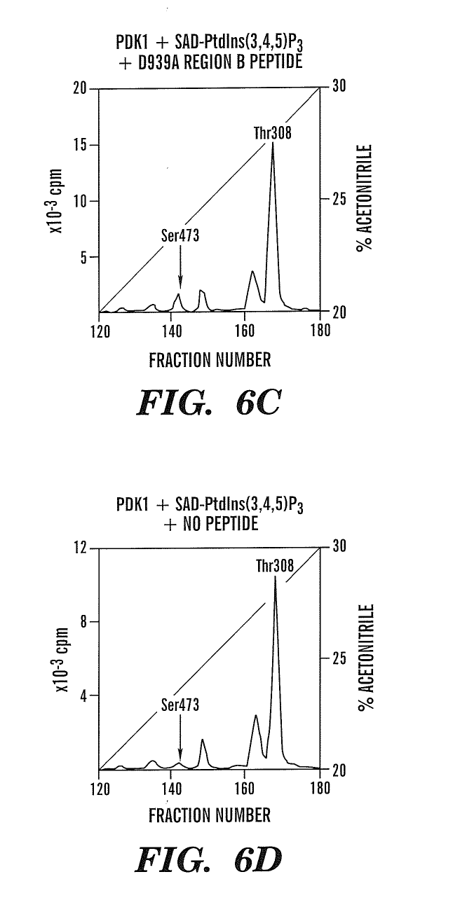

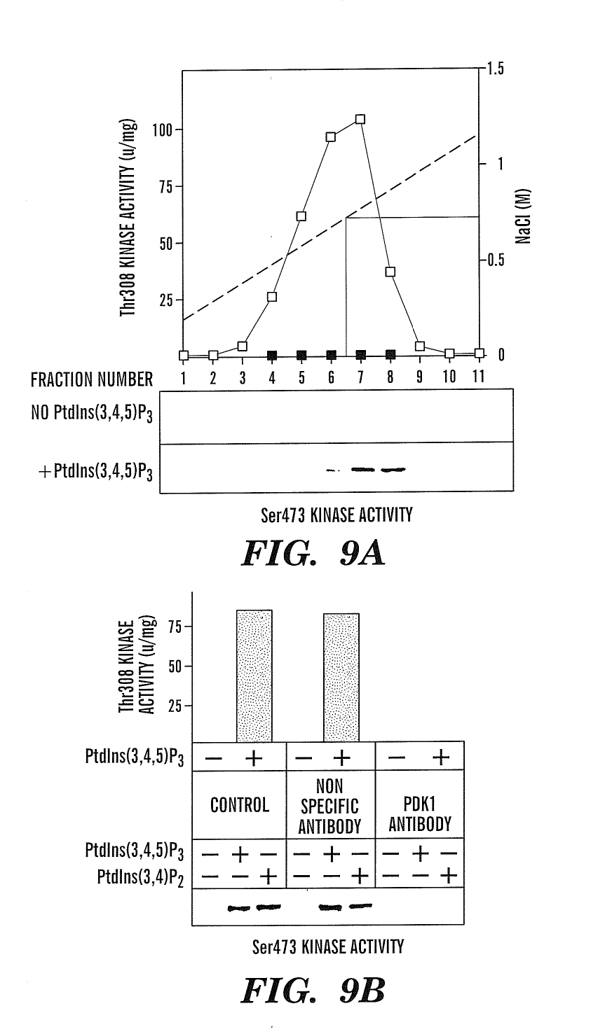

Synthetic sn-1-stearoyl, 2-arachidonoyl D-PtdIns(3,4,5,)P₃ (the predominant form of PtdIns(3,4,5)P₃ which occurs naturally, lipid 8 in FIG. 9), synthetic sn-2-stearoyl, 3-arachidonoyl D-PtdIns(3,4,5,)P₃ (lipid 10 in FIG. 9) may be capable of enhancing the phosphorylation by PDK1 (after said exposure) of GST-PKBα at Ser473 (FIG. 9B) and the phosphorylation of Thr308 (measured by the activation of GST-S473D-PKBα), indicating that the enantiomeric configuration of the glycerol moiety is not an important determinant of specificity. The L-enantiomers of these lipids (Lipids 9 and 11, FIG. 9) may not induce a significant phosphorylation of PKB at Ser473 or activation of GST-S473D-PKBα. rac-1,2-dilinoleoyl phosphatidyl D/L-myo-inositol 3,4,5 trisphosphate (linoleic acid is C18:2, Lipid 7 FIG. 9), sn-1,2-dipalmitoyl D-PtdIns(3,4,5)P₃, (Lipid 6, FIG. 6) as well as sn-1,2-dipalmitoyl PtdIns(3,4)P₂ (Lipid 3 FIG. 9) may also be effective at inducing the PDK1 (after said exposure) to phosphorylate PKBα at Ser473 and Thr308. However, PtdIns-3P (lipid 2, FIG. 9), PtdIns(3,5)P₂ (lipid 4 FIG. 9) and PtdIns(4,5)P₂ (lipid 5 FIG. 9), did not induce the PDK1 (after said exposure) to phosphorylate PKBα at Ser473 or Thr308. In the absence of GST-PIF none of the PtdIns derivatives tested induced any phosphorylation of Ser473 or activation of GST-S473D-PKBα (data not shown).

The said interacting polypeptide may be derivable from PRK2 or PKCζ, preferably from the C-terminal portion of PRK2 or PKCζ. The said interacting polypeptide may be derivable from PRK2 by proteolytic cleavage, for example by Caspase 3, as described in Example 1.

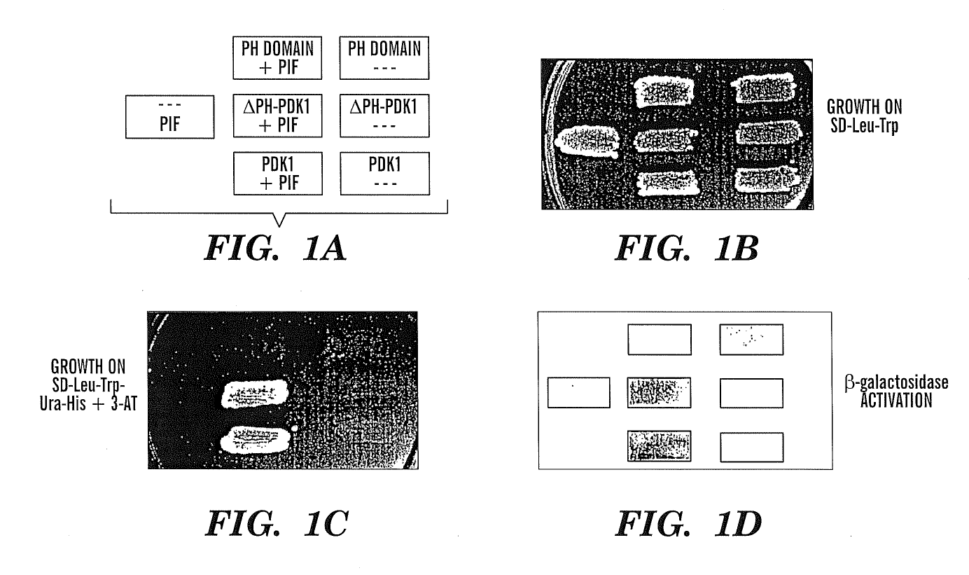

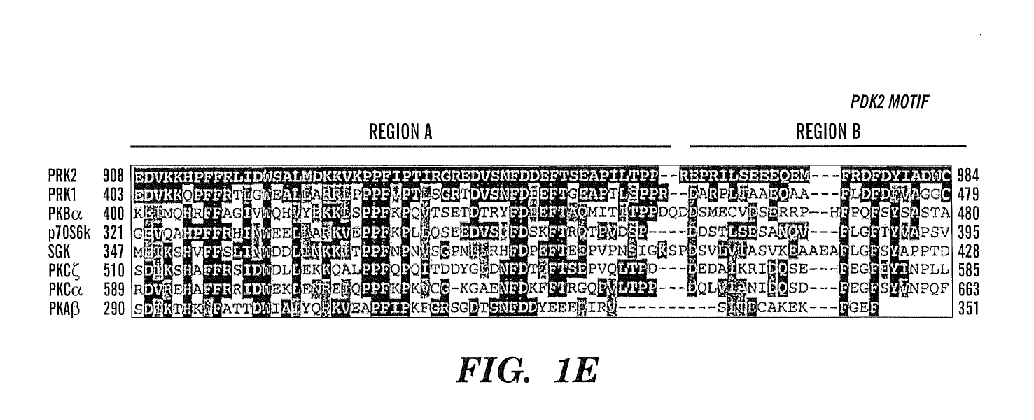

Thus, the interacting polypeptide may comprise or consist essentially of the amino acid sequence from residue 701 to the C-terminus of PRK2. This may correspond to the C-terminal 77 amino acids of PRK2. The C-terminal 77 amino acids of PRK2 may be termed the PDK1-Interacting Fragment (PIF). The PIF region of PRK2 may lie immediately C-terminal to the kinase catalytic domain of PRK2. The polypeptide may comprise or consist essentially of the amino acid sequence of residues 960 to 984 of PRK2 (termed Region B, as described in Example 1) or the equivalent region of PRK1, PRK1, PKBα, p70S6 kinase, SGK, a PKC isoform, for example PKCζ or PKCα, or PKAβ as shown in FIG. 1E. PKC isoforms are described, for example, in Mellor & Parker (1998) The extended protein kinase C superfamily Biochem J 332, 281-292. The PIF region and Region B of PRK2 comprise an amino acid sequence corresponding to the consensus sequence Phe/Tyr-Xaa-Xaa-Phe/Tyr-Zaa-Phe/Tyr (SEQ ID NO:25) or Phe/Tyr-Xaa-Xaa-Phe/Tyr-Asp/Glu-Phe/Tyr (SEQ ID NO:26). The C-terminal region of PKCζ comprises the amino acid sequence Phe-Glu-Gly-Phe-Glu-Tyr (SEQ ID NO:29), which corresponds to the consensus sequence Phe/Tyr-Xaa-Xaa-Phe/Tyr-Zaa-Phe/Tyr (SEQ ID NO:25) or Phe/Tyr-Xaa-Xaa-Phe/Tyr-Asp/Glu-Phe/Tyr (SEQ ID NO:26). It will be appreciated that a polypeptide that comprises an amino acid sequence that corresponds to the consensus sequence Phe/Tyr-Xaa-Xaa-Phe/Tyr-Ser/Thr-Phe/Tyr (SEQ ID NO:30) may interact with PDK1 when the serine or threonine residue is phosphorylated, such that the polypeptide comprises an amino acid sequence that corresponds to the consensus sequence Phe/Tyr-Xaa-Xaa-Phe/Tyr-PhosphoSer/PhosphoThr-Phe/Tyr (SEQ ID NO:27), or if the serine or threonine residue is replaced by an aspartate or glutamate residue.

Region B of PRK2 may have the amino acid sequence REPRILSEEEQEMFRDFDYIADWC (SEQ ID NO:1). Thus, the said interacting polypeptide to which the said PDK1 may be exposed may comprise or consist essentially of the sequence REPRILSEEEQEMFRDFDYIADWC (SEQ ID NO:1) or REPRILSEEEQEMARDFDYIADWC (SEQ ID NO:2) or REPRILSEEEQEMFGDFDYIADWC (SEQ ID NO:3).

Region A of PRK2 may have the amino acid sequence EDVKKHPFFRLIDWSALMDKKVKPPFIPTIRGREDVSNFDDEFTSEAPILTPP (SEQ ID NO:4). Thus, the said interacting polypeptide may further comprise the sequence EDVKKHPFFRLIDWSALMDKKVKPPFIPTIRGREDVSNFDDEFTSEAPILTPP (SEQ ID NO:4) or variants thereof.

The region of PKCζ equivalent to Region B of PRK2 may have the amino acid sequence DEDAIKRIDQSEFEGFEYINPLL (SEQ ID NO:5), as shown in FIG. 1. Thus, the said interacting polypeptide may comprise the sequence DEDAIKRIDQSEFEGFEYINPLL (SEQ ID NO:5) or variants thereof.

The said interacting polypeptide may comprise a GST portion, as described in Example 1. This may be useful in purifying and/or detecting the said interacting polypeptide.

The said PDK1 and said interacting polypeptide may be exposed to each other in a cell in which the said PDK1 and the said interacting polypeptide are both expressed, as described in Example 1. The PDK1 may be endogenous PDK1 or it may be PDK1 expressed from a recombinant construct. Similarly, the said interacting polypeptide may be endogenous or it may be expressed from a recombinant construct, as described in Example 1. It is preferred that the said PDK1 and the said interacting polypeptide are not exposed to each other in a cell in which the said PDK1 and the said interacting polypeptide are both naturally expressed. It is preferred that the said PDK1 and the said interacting polypeptide are not both endogenous polypeptides to the cell in which the said PDK1 and the said interacting polypeptide are exposed to each other.

The PDK1 and said polypeptide may form a complex, which may be detected by BiaCore measurements, as described in Example 1. The estimated equilibrium dissociation constant of the association between GST-PIF and His-tagged PDK1 may be 600 nM. The estimated dissociation constant K_{d} between His-PDK1 and an immobilised and biotinylated 24 residue synthetic peptide corresponding to Region B above detected using Surface Plasmon Resonance measurements was 800 nM, or 1.5 μM. A complex may also be detected by coimmunoprecipitation or copurification experiments, for example in material from cells in which PDK1 (as defined above) and the said polypeptide are coexpressed, as described in Example 1. The complex between PDK1 (for example full-length wild type PDK1) and the said polypeptide (for example, GST-PIF) may be a very strong interaction, as indicated by not being dissociated by up to 2M LiBr (a strong chaotrophic agent) or by incubation with 1% (by vol) Triton X100.

A further aspect of the invention provides a form of PDK1 derivable by the method of the first aspect of the invention wherein the said PDK1 has altered substrate specificity. As described above, the said PDK1 with altered substrate specificity may be capable of phosphorylating the underlined residue in a polypeptide with an amino acid sequence corresponding to the consensus sequence Phe-Xaa-Xaa-Phe-Ser/Thr-Phe/Tyr (SEQ ID NO:24). The said PDK1 may be substantially free of polypeptides with which PDK1 is present or associated in a cell in which it is naturally found other than a said interacting polypeptide or a substrate for PDK1.

A further aspect of the invention provides a method of making a preparation that comprises PDK1 and a polypeptide which comprises the amino acid sequence Phe/Tyr-Xaa-Xaa-Phe/Tyr-Zaa-Phe/Tyr (SEQ ID NO:25) wherein substantially pure PDK1 (as defined above) is mixed with a substantially pure polypeptide which comprises the amino acid sequence Phe/Tyr-Xaa-Xaa-Phe/Tyr-Zaa-Phe/Tyr (SEQ ID NO:25). It will be appreciated that the method may further comprise the step of adding further components to the preparation, for example a stabilizing component such as bovine serum albumin or a substrate of the said PDK1, for example PKBα.

A further aspect of the invention provides a preparation derivable by the above method of the invention.

By “substantially pure” we mean that the PDK1 or interacting polypeptide is substantially free of other proteins. Thus, we include any composition that includes at least 30% of the protein content by weight as the said PDK1 or interacting polypeptide, preferably at least 50%, more preferably at least 70%, still more preferably at least 90% and most preferably at least 95% of the protein content is the said PDK1 or interacting polypeptide.

Thus the substantially pure PDK1 or interacting polypeptide may include a contaminant wherein the contaminant comprises less than 70% of the composition by weight, preferably less than 50% of the composition, more preferably less than 30% of the composition, still more preferably less than 10% of the composition and most preferably less than 5% of the composition by weight.

The substantially pure said PDK1 or interacting polypeptide may be combined with other components ex vivo, said other components not being all of the components found in the cell in which said PDK1 or interacting polypeptide is naturally found.

A further aspect of the invention provides a preparation comprising PDK1 and a polypeptide which comprises the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27) (the interacting polypeptide), wherein the said preparation does not comprise all of the components found in a cell in which said PDK1 is naturally found. The said preparation may not comprise components found in a cell, for example a rat brain cell, that are (1) not capable of binding to a Q-Sepharose column equilibrated in buffer solution containing 50 mMTris/HCl pH7.5, 1 mM EDTA, 1 mM EGTA, 0.27M sucrose and 0.1% (by vol) β-mercaptoethanol, or (2) capable of binding to a Q-Sepharose column equilibrated in buffer solution containing 50 mMTris/HCl pH7.5, 1 mM EDTA, 1 mM EGTA, 0.27M sucrose and 0.1% (by vol) β-mercaptoethanol but are not eluted by the said buffer solution further containing 0.3M NaCl. The said preparation may further not comprise components found in a cell that are (1) not capable of binding to a heparin-Sepharose column equilibrated in buffer solution containing 50 mMTris/HCl pH7.5, 1 mM EDTA, 1 mM EGTA, 0.27M sucrose, 0.1% (by vol) β-mercaptoethanol and 0.2M NaCl or (2) capable of binding to a heparin-Sepharose column equilibrated in buffer solution containing 50 mMTris/HCl pH7.5, 1 mM EDTA, 1 mM EGTA, 0.27M sucrose, 0.1% (by vol) β-mercaptoethanol and 0.2M NaCl but are eluted by the said buffer solution containing up to 0.75M NaCl. Thus, the preparation may be substantially free of polypeptides with which PDK1 is present or associated in a cell other than a said interacting polypeptide or a substrate of PDK1, for example PKBα.

The said preparation may not be obtainable by (1) homogenising rat tissue (fore example brain or thymus) in 20 mM Tris pH7.5, 1 mM EDTA, 25 mM NaF, 1 mM dithiothreitol (DTT), 1 mM NaVn, leupeptin (10 μg/ml), soybean trypsin inhibitor (10 μg/ml), aprotinin (10 μg/ml) and 100 μM pefabloc, (2) centrifuging at 20,000 g for 30 min, (3) loading extracts (for example, 20 mg) onto a Mono Q column (Pharmacia) and (4) eluting the bound proteins with up to 250 mM NaCl, as described in Stokoe et al (1997) Science 277, 567-570.

Thus, we include any composition that includes at least 30% of the protein content by weight as the said PDK1 or interacting polypeptide (ie in combination), preferably at least 50%, more preferably at least 70%, still more preferably at least 90% and most preferably at least 95% of the protein content is the said PDK1 or interacting polypeptide.

Thus, the invention also includes preparations comprising the said PDK1 and the said interacting polypeptide and a contaminant wherein the contaminant comprises less than 70% of the composition by weight, preferably less than 50% of the composition, more preferably less than 30% of the composition, still more preferably less than 10% of the composition and most preferably less than 5% of the composition by weight. The invention also includes a preparation comprising the said PDK1 and the said interacting polypeptide when combined with other components ex vivo, said other components not being all of the components found in the cell in which said PDK1 and/or interacting polypeptide is naturally found.

Alternatives and preferences for PDK1 and the said interacting polypeptide are as described in relation to the first aspect of the invention.

A further aspect of the invention provides a preparation capable of phosphorylating the underlined residue in a polypeptide with an amino acid sequence corresponding to the consensus sequence Phe-Xaa-Xaa-Phe-Ser/Thr-Phe/Tyr (SEQ ID NO:24), wherein said preparation comprises two, three, four, five, six or up to ten species of polypeptide and substantially (by mass) no other polypeptides. One said polypeptide species is PDK1 and a second said polypeptide species (the interacting polypeptide) comprises the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27). Alternatives and preferences for PDK1 and the said interacting polypeptide are as described in relation to the first aspect of the invention. A third polypeptide species may be a substrate for PDK1. A fourth polypeptide species may be a polypeptide that stabilizes the preparation, for example bovine serum albumin or gelatin.

It is preferred that the said preparation is capable of phosphorylating a residue corresponding to the underlined residue in a polypeptide with an amino acid sequence corresponding to the consensus sequence Thr/Ser-Phe-Cys-Gly-Thr-Xaa-Glu-Leu (SEQ ID NO:28).

It will be appreciated that the two, three, four, five, six or up to ten species of polypeptide may be capable of forming an isolatable complex, as known to those skilled in the art and discussed in Example 1 in relation to PDK1 and a polypeptide comprising the amino acid sequence Phe/Tyr-Xaa-Xaa-Phe/Tyr-Zaa-Phe/Tyr (SEQ ID NO:25).

A further aspect of the invention provides a method of phosphorylating a residue corresponding to the underlined residue in a substrate polypeptide with an amino acid sequence corresponding to the consensus sequence Phe-Xaa-Xaa-Phe-Ser/Thr-Phe/Tyr (SEQ ID NO:24) (PDK2 consensus sequence) wherein a PDK1 derivable by the method of the first aspect of the invention, characterised in that the said PDK1 has altered substrate specificity, or a preparation according to either of the preceding aspects of the invention is used.

The substrate polypeptide may be PKB, for example PKBα, SGK, p70S6 kinase, PKA or a PKC isoform. It is preferred that the substrate polypeptide is also phosphorylated on a residue corresponding to the underlined residue in the consensus sequence Thr/Ser-Phe-Cys-Gly-Thr-Xaa-Glu-Leu (SEQ ID NO:28).

It will be appreciated that the method may be carried out in the presence of a phosphoinositide, for example PIP₂ or PtdIns(3,4,5)P₃ (PIP₃). The said PIP₂ or PIP₃ may increase the rate or extent of phosphorylation of the underlined residue in a substrate polypeptide with an amino acid sequence corresponding to the consensus sequence Phe-Xaa-Xaa-Phe-Ser/Thr-Phe/Tyr (SEQ ID NO:24) PDK2 consensus sequence and/or of the residue corresponding to the underlined residue in the consensus sequence Thr/Ser-Phe-Cys-Gly-Thr-Xaa-Glu-Leu (SEQ ID NO:28).

A further aspect of the invention provides a method of phosphorylating PRK2 wherein the said PRK2 is exposed to PDK1. PRK2 may be phosphorylated on the residue corresponding to Thr807 of full-length PRK2. A further aspect of the invention provides the use of PDK1 in a method of phosphorylating PRK2.

A further aspect of the invention provides a method of identifying a compound that modulates the activation and/or phosphorylation of PRK2 by PDK1 wherein the activation and/or phosphorylation of PRK2 by PDK1 is measured in the presence of more than one concentration (for example in the presence or absence) of the compound.

A still further aspect of the invention provides a preparation comprising PDK1 and PRK2 in the substantial absence of other proteins or cellular components of a cell in which said PDK1 and/or PRK2 are naturally found. Preferences for this preparation of the invention are analogous to the preferences described above for the preparation of the invention comprising PDK1 and a polypeptide which comprises the amino acid sequence Phe/Tyr-Xaa-Xaa-Phe/Tyr-Zaa-Phe/Tyr (SEQ ID NO:25) wherein the said preparation does not comprise all of the components found in a cell in which said PDK1 and/or interacting polypeptide is naturally found.

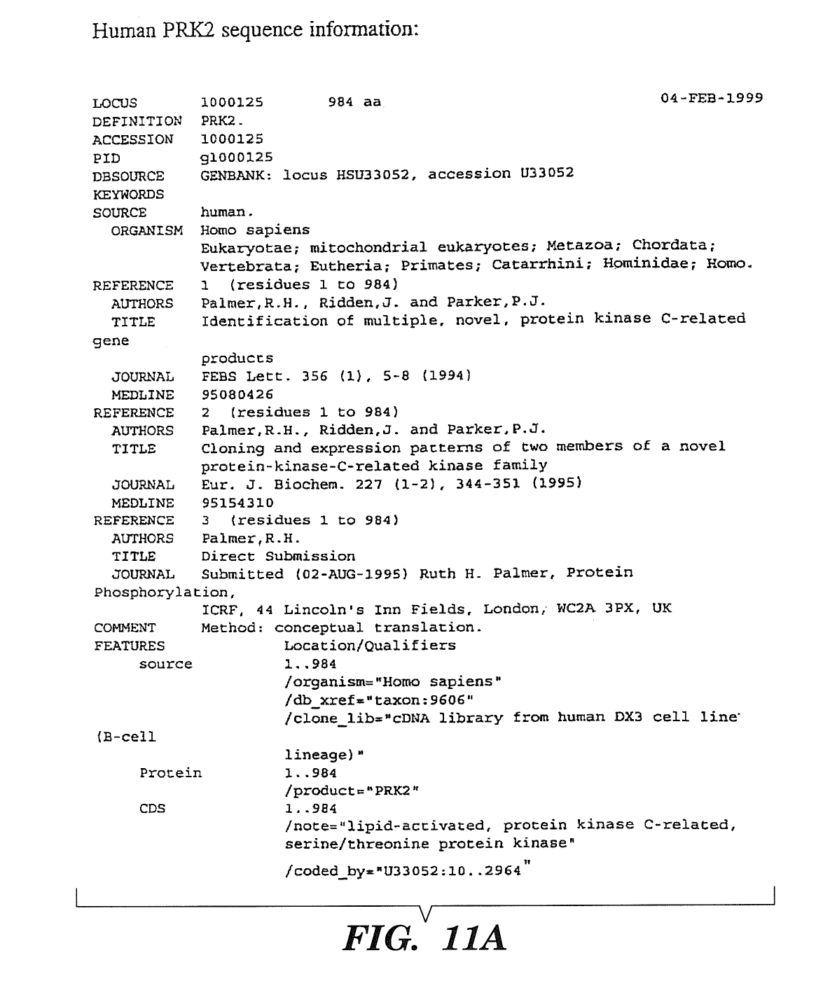

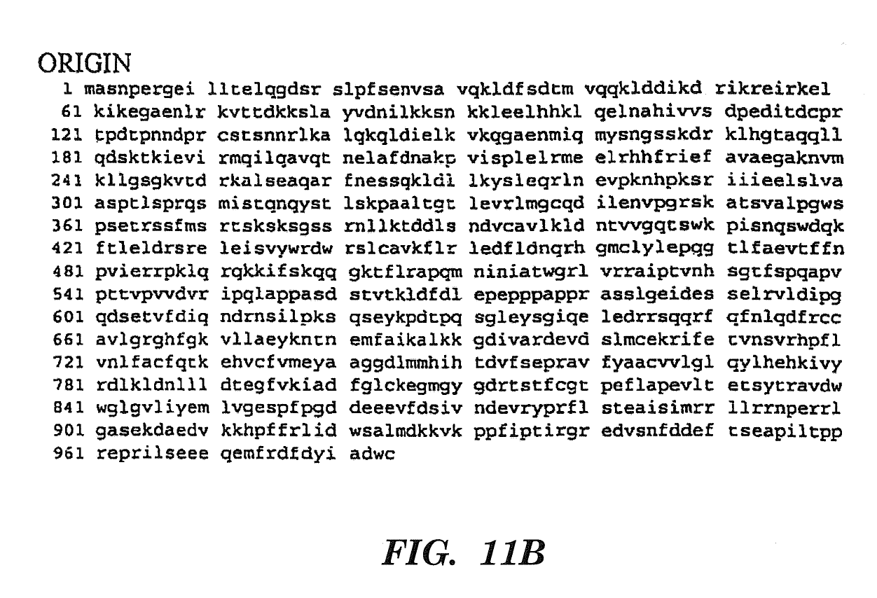

By PRK2 is included a polypeptide with the amino acid sequence shown in FIG. 11 or given in Palmer et al (1994) Identification of multiple, novel, protein kinase C-related gene products FEBS Lett 356(1), 5-8, and fragments, variants, derivatives and fusions thereof.

A further aspect of the invention provides a method of identifying a compound that modulates the activity of PDK1 wherein the said PDK1 is exposed to the said compound in the presence of a polypeptide comprising the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27). By activity of PDK1 is included the capability of phosphorylating the underlined residue in a substrate polypeptide with an amino acid sequence corresponding to the consensus sequence Phe-Xaa-Xaa-Phe-Ser/Thr-Phe/Tyr (SEQ ID NO:24) (ie PDK2 activity) and/or the capability of phosphorylating the residue corresponding to the underlined residue in the consensus sequence Thr/Ser-Phe-Cys-Gly-Thr-Xaa-Glu-Leu (SEQ ID NO:28) (ie PDK1 activity).

The method of identifying a compound that modulates the activity of PDK1 may comprise the step of measuring the activity of the said PDK1 in the presence of more than one concentration of the compound (for example, in the presence of the compound and in the presence of substantially none of the compound) wherein the said PDK1 is or has been exposed to a polypeptide which comprises the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27).

The said compound may be capable of modulating the interaction between the said polypeptide and PDK1. It will be appreciated that the said compound may interact with PDK1 or with the said polypeptide or with both.

A further aspect of the invention provides a method of identifying a compound that is capable of altering the substrate specificity of PDK1 wherein the ability of the said PDK1 to phosphorylate a residue corresponding to the underlined residue in a polypeptide with an amino acid sequence corresponding to the consensus sequence Phe-Xaa-Xaa-Phe-Ser/Thr-Phe/Tyr (SEQ ID NO:24) is measured, and is increased in the presence of the said compound. It will be appreciated that the said ability of PDK1 may be measured in the presence of a phosphoinositide, for example PtdIns(3,4,5)P₃ or PtdIns(3,4)P₂. A further aspect of the invention is a compound identified or identifiable by the said method.

The compound may be, for example, a compound selected on the basis of, or designed to have, as well known to those skilled in the art, a three-dimensional conformation that may be similar to that of a polypeptide with or comprising an amino acid sequence corresponding to the consensus sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26), in particular a polypeptide with the sequence FRDFDY (SEQ ID NO:6) or REPRILSEEEQEMFRDFDYIADWC (SEQ ID NO:1).

A further aspect of the invention is a method of altering the substrate specificity of phosphoinositide-dependent protein kinase 1 (PDK1) wherein the said PDK1 is exposed to a compound identified or identifiable by the above method of the invention. A further aspect of the invention is a PDK1 derivable by the method of the above aspect of the invention wherein the said PDK1 has altered substrate specificity such that the said PDK1 is capable of phosphorylating a residue corresponding to the underlined residue in a polypeptide having an amino acid sequence corresponding to the consensus sequence Phe/Tyr-Xaa-Xaa-Phe/Tyr-Ser/Thr-Phe/Tyr (SEQ ID NO:30).

A further aspect of the invention provides a method of identifying a compound that is capable of mimicking the effect of a 3-phosphoinositide, for example PtdIns(3,4,5)P₃ or PtdIns(3,4)P₂, on the PDK1 or PDK2 activity of a PDK1 which has altered substrate specificity as defined above, the method comprising determining whether said compound activates a said PDK1 so that it can phosphorylate a suitable substrate, the activation by said compound being in the absence of a 3-phosphoinositide. The suitable substrate may be a polypeptide comprising an amino acid sequence corresponding to the consensus sequence Phe-Xaa-Xaa-Phe-Ser/Thr-Phe/Tyr (SEQ ID NO:24) (for measuring PDK2 activity) or a polypeptide comprising an amino acid sequence corresponding to the consensus sequence Thr/Ser-Phe-Cys-Gly-Thr-Xaa-Glu-Leu (SEQ ID NO:28) (for measuring PDK1 activity).

A further aspect of the invention provides a protein kinase derivable from mammalian, for example rat, brain wherein said protein kinase is capable of phosphorylating a residue corresponding to the underlined residue in a polypeptide with an amino acid sequence corresponding to the consensus sequence Phe-Xaa-Xaa-Phe-Ser/Thr-Phe/Tyr (SEQ ID NO:24), for example Ser473 of PKBα in the presence of PtdIns(3,4,5)P₃, wherein the said protein kinase may be eluted from Heparin-Sepharose by at least 0.75M NaCl at pH 7.5 and is capable of binding to an antibody reactive with PDK1. Further details of a method of purifying the said protein kinase are given in Example 1. It will be appreciated that the said protein kinase may comprise more than one polypeptide chain, for example in a non-covalently bound complex.

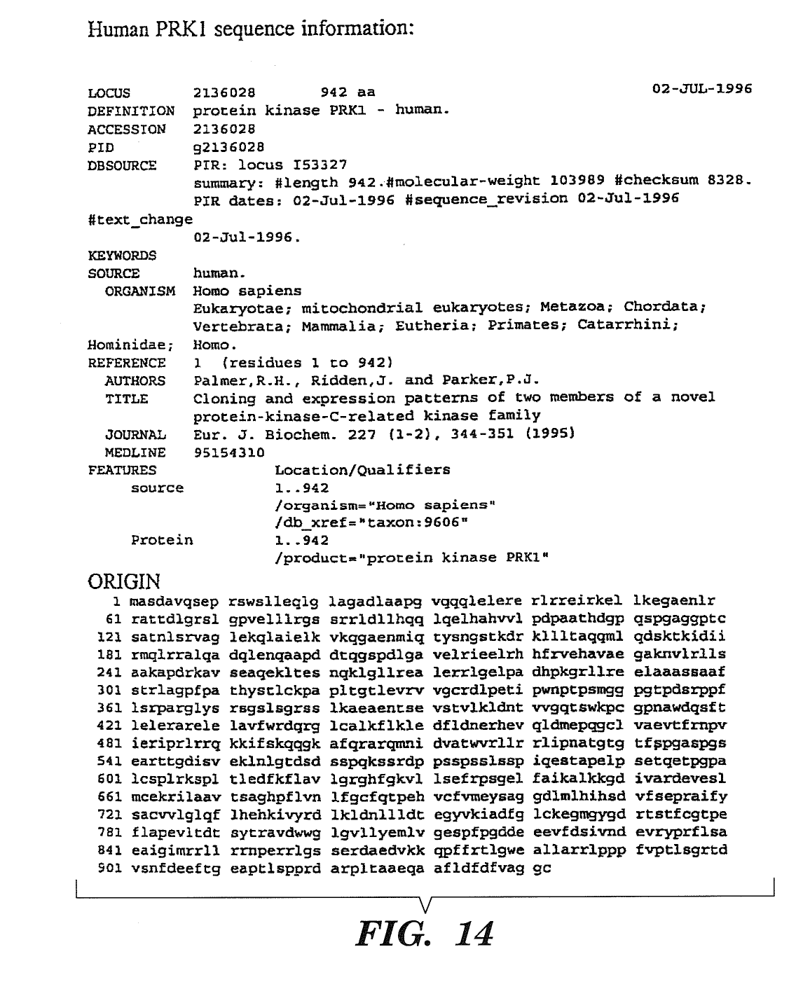

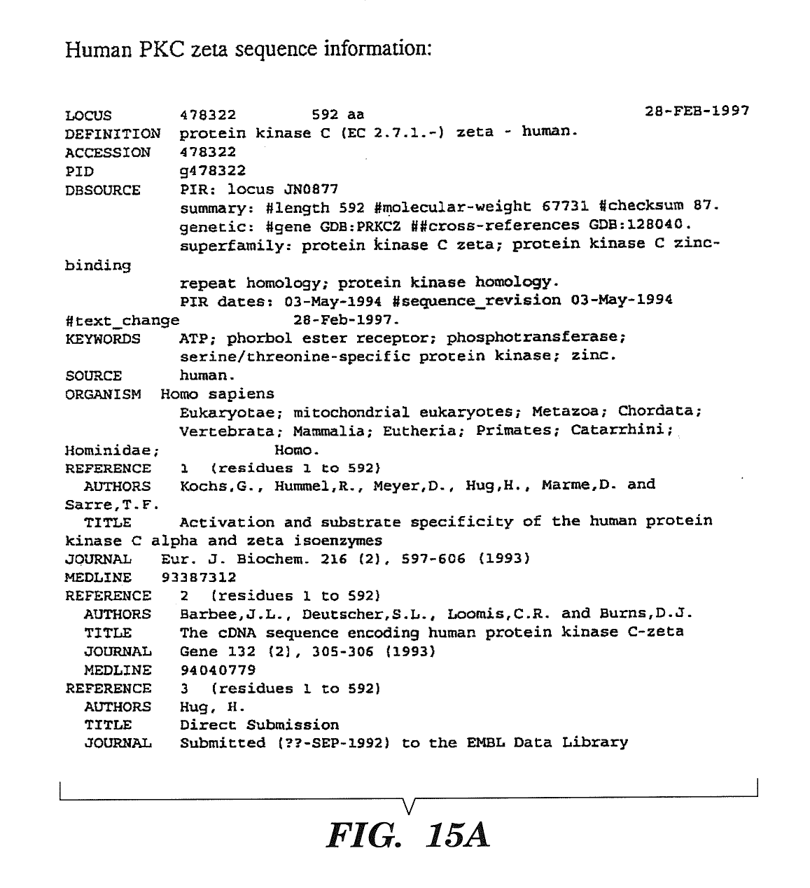

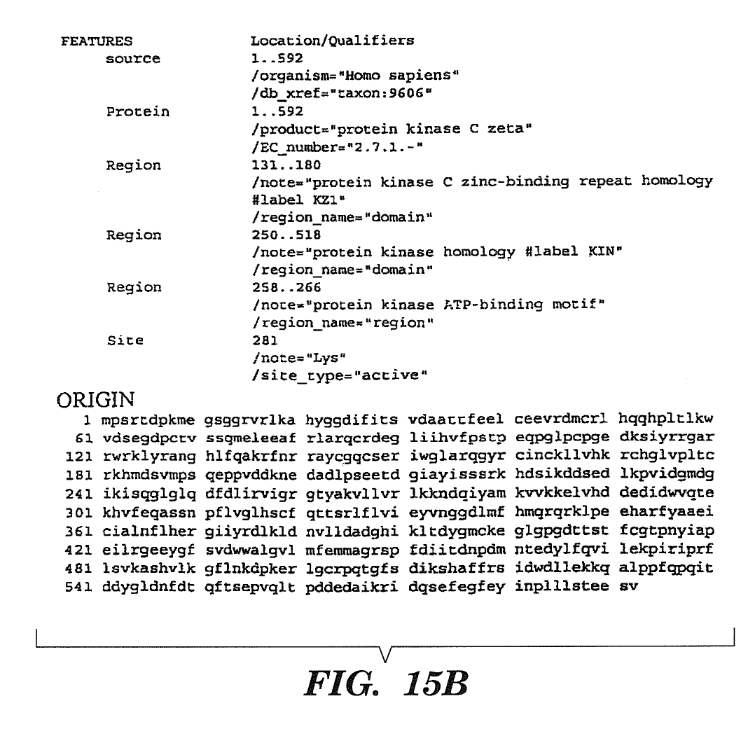

A further aspect of the invention provides a polypeptide which comprises the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26), wherein said polypeptide is not full-length PRK2, PRK1 or PKCζ and wherein Zaa is not phosphoserine or phosphothreonine. The sequence of PRK2 is shown in FIG. 11 (SEQ ID NO:32) and in Palmer et al (1994) Identification of multiple, novel, protein kinase C-related gene products FEBS Lett 356(1), 5-8. The sequence of PRK1 is shown in FIG. 14 (SEQ ID NO:33) and in Palmer et al (1995) Cloning and expression patterns of two members of a novel protein-kinase-C-related kinase family Eur J Biochem 227(1-2), 344-351. The sequence of PKCζ is shown in FIG. 15 (SEQ ID NO:34) and in Kochs et al (1993) Activation and substrate specificity of the human protein kinase C alpha and zeta isoenzymes Eur J Biochem 216(2), 597-606. Thus, the polypeptide may comprise or consist essentially of the amino acid sequence from residue 701 to the C-terminus of PRK2. This may correspond to the C-terminal 77 amino acids of PRK2. The C-terminal 77 amino acids of PRK2 may be termed the PDK1-Interacting Fragment (PIF). The PIF region of PRK2 may lie immediately C-terminal to the kinase catalytic domain of PRK2. 21. The polypeptide may comprise or consist essentially of the amino acid sequence of residues 960 to 984 of PRK2 (termed Region B, as described in Example 1). The PIF region and Region B of PRK2 comprise an amino acid sequence corresponding to the consensus sequence Phe/Tyr-Xaa-Xaa-Phe/Tyr-Asp/Glu-Phe/Tyr (SEQ ID NO:26).

Region A may have the amino acid sequence REPRILSEEEQEMFRDFDYIADWC (SEQ ID NO:1). Thus, the said polypeptide of the invention may comprise or consist essentially of the sequence REPRILSEEEQEMFRDFDYIADWC (SEQ ID NO:1)or REPRILSEEEQEMARDFDYIADWC (SEQ ID NO:2) or REPRILSEEEQEMFGDFDYIADWC (SEQ ID NO:3).

Region B may have the amino acid sequence EDVKKHPFFRLIDWSALMDKKVKPPFIPTIRGREDVSNFDDEFTSEAPILTPP (SEQ ID NO:4). Thus, the said polypeptide may further comprise the sequence EDVKKHPFFRLIDWSALMDKKVKPPFIPTIRGREDVSNFDDEFTSEAPILTPP (SEQ ID NO:4) or variants thereof. The said polypeptide of the invention may comprise a GST portion, as described in Example 1. This may be useful in purifying and/or detecting the said polypeptide.

A further aspect of the invention provides a polypeptide consisting essentially of residues 51 to 404 of PDK1 or a fusion of a polypeptide consisting essentially of residues 51 to 404 of PDK1.

A further aspect of the invention provides a polynucleotide encoding a polypeptide of the invention. A still further aspect of the invention provides a recombinant polynucleotide suitable for expressing a polypeptide of the invention. A yet further aspect of the invention provides host cell comprising a polynucleotide of the invention.

A further aspect of the invention provides a method of making a polypeptide of the invention, the method comprising culturing a host cell of the invention which expresses said polypeptide and isolating said polypeptide. It will be appreciated that the said polypeptide of the invention that comprises the amino acid sequence Phe/Tyr-Xaa-Xaa-Phe/Tyr-Asp/Glu-Phe/Tyr (SEQ ID NO:26) may be isolated as a complex with endogenous PDK1 expressed in the cell or with recombinant PDK1 expressed in the cell. It will be appreciated that the said polypeptide of the invention that consists essentially of residues 51 to 404 of PDK1 or a fusion of a polypeptide consisting essentially of residues 51 to 404 of PDK1 may be isolated as a complex with endogenous PRK2 or a fragment thereof, as described above and in Example 1, or with recombinant PRK2 or a fragment, derivative or fusion thereof as described in Example 1 expressed in the cell.

A further aspect of the invention provides a polypeptide obtainable by the above method. A further aspect of the invention provides a polypeptide of the invention for use in medicine. A still further aspect of the invention provides the use of a polypeptide of the invention in the manufacture of a medicament for the treatment of a patient in need of modulation of the insulin signalling pathway and/or PDK1/PDK2/PRK2 signalling.

The polypeptide of the invention or interacting polypeptide which comprises the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27) as defined above may be up to about 950, 900, 800, 700, 600, 500, 400, 300, 200, 100, 80, 70, 60, 50, 40, 30, 20, 18, 16, 15, 14, 12, 10, 8 or 7 amino acids in length. The polypeptide may consist of or comprise contiguous residues derivable from PRK2, PKCζ or PDK1 (as appropriate), for example rat or human PRK2, PKCζ or PDK1. The polypeptide may be capable of reducing, preferably substantially preventing, an interaction between full length PDK1, for example full length human PDK1 and a polypeptide comprising the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoThr/PhosphoSer-Phe/Tyr (SEQ ID NO:27), measured, for example, as described in Example 1. It will be appreciated that the peptide may comprise a covalent modification, for example it may be modified by biotinylation ie comprise a biotin group. Such a peptide may be useful in altering the enzymic activity of PDK1 in vitro or in vivo.

The above polypeptides or peptide may be made by methods well known in the art and as described below and in Example 1, for example using molecular biology methods or automated chemical peptide synthesis methods.

It will be appreciated that peptidomimetic compounds may also be useful. Thus, by “polypeptide” or “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Mézière et al (1997) J. Immunol. 159, 3230-3237, incorporated herein by reference. This approach involves making pseudopeptides containing changes involving the backbone, and not the orientation of side chains. Mézière et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the Cl atoms of the amino acid residues is used; it is particularly preferred if the linker moiety has substantially the same charge distribution and substantially the same planarity as a peptide bond.

It will be appreciated that the peptide may conveniently be blocked at its N- or C-terminus so as to help reduce susceptibility to exoproteolytic digestion.

Thus, it will be appreciated that the polypeptide which comprises the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27) to which PDK1 may be exposed may be a peptidomimetic compound, as described above.

A further aspect of the invention is a cell containing a recombinant nucleic acid suitable for expressing PDK1 and a recombinant nucleic acid suitable for expressing a polypeptide comprising the amino acid sequence Phe/Tyr-Xaa-Xaa-Phe/Tyr-Zaa-Phe/Tyr (SEQ ID NO:25). Recombinant polynucleotides suitable for expressing a given polypeptide are well known to those skilled in the art, and examples are described in Example 1. It will be appreciated that a recombinant nucleic acid molecule may be suitable for expressing PDK1 and a polypeptide comprising the amino acid sequence Phe/Tyr-Xaa-Xaa-Phe/Tyr-Zaa-Phe/Tyr (SEQ ID NO:25). The cell is preferably a mammalian or insect cell.

A further aspect of the invention is a method of altering the substrate specificity of PDK1 according to the first aspect of the invention wherein PDK1 is exposed to an interacting polypeptide comprising the amino acid sequence Phe/Tyr-Xaa-Xaa-Phe/Tyr-Zaa-Phe/Tyr (SEQ ID NO:25) in a cell as defined in the above aspect of the invention, by co-expression of PDK1 and the said interacting polypeptide. A further aspect of the invention is a method of making a preparation comprising PDK1 and an interacting polypeptide comprising the amino acid sequence Phe/Tyr-Xaa-Xaa-Phe/Tyr-Zaa-Phe/Tyr (SEQ ID NO:25) wherein PDK1 and the said interacting polypeptide are co-expressed in a cell as defined in the above aspect of the invention. The PDK1 and the said interacting polypeptide may be separated from other cellular components, for example using methods discussed above or in Example 1. A further aspect of the invention is a preparation obtainable by the above method of the invention.

An antibody reactive towards a polypeptide with the amino acid sequence REPRILSEEEQEMFRDFDYIADWC (SEQ ID NO:1) or REPRILSEEEQEMARDFDYIADWC (SEQ ID NO:2) or REPRILSEEEQEMFGDFDYIADWC (SEQ ID NO:3) is described in example 1.

An antibody reactive towards PKBα or a fragment or fusion thereof that is phosphorylated on the residue equivalent to Ser473 but is not reactive with PKBα or a fragment or fusion thereof that is not phosphorylated on the residue equivalent to Ser473 is described in Example 1. The antibody may react with the peptide Pro-His-Phe-Pro-Gln-Phe-PhosphoSer-Tyr-Ser-Ala-Ser (SEQ ID NO:8) (corresponding to residues 467 to 477 of PKBα).

Methods of preparing such antibodies are given in Example 1.

Antibodies reactive towards the said polypeptides may be made by methods well known in the art. In particular, the antibodies may be polyclonal or monoclonal.

Suitable monoclonal antibodies which are reactive towards the said polypeptide may be prepared by known techniques, for example those disclosed in “Monoclonal Antibodies: A manual of techniques”, H Zola (CRC Press, 1988) and in “Monoclonal Hybridoma Antibodies: Techniques and Applications”, SGR Hurrell (CRC Press, 1982).

Techniques for preparing antibodies are well known to those skilled in the art, for example as described in Harlow, ED & Lane, D “Antibodies: a laboratory manual” (1988) New York Cold Spring Harbor Laboratory.

The enhancement or disruption of the interaction between PDK1 and an interacting polypeptide can be measured in vitro using methods well known in the art of biochemistry and include any methods which can be used to assess protein-protein interactions. It will be appreciated that the methods described may be performed in cells. In a further embodiment the yeast two-hybrid system may be used.

It will be appreciated that the invention provides screening assays for drugs which may be useful in modulating, for example either enhancing or inhibiting, the PDK1 or PDK2 activity (as discussed above) of PDK1, for example PDK1 that has been exposed to a polypeptide comprising the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27), or the interaction of PDK1 with a phosphoinositide, for example PtdIns(3,4,5)P₃ or PtdIns(3,4)P₂, or the interaction of PDK1 with a polypeptide comprising the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27). The compounds identified in the methods may themselves be useful as a drug or they may represent lead compounds for the design and synthesis of more efficacious compounds.

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons. A drug-like compound may additionally exhibit features of selective interaction with a particular protein or proteins and be bioavailable and/or able to penetrate cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

A further aspect of the invention is a kit of parts useful in carrying out a method, for example a screening method, of the invention. Such a kit may comprise PDK1 and a polypeptide comprising the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27). It may further comprise a 3-phosphoinositide, for example PtdIns(3,4,5)P₃ or PtdIns(3,4)P₂.

It will be understood that it will be desirable to identify compounds that may modulate the activity of the PDK1 polypeptide in vivo. Thus it will be understood that reagents and conditions used in the method may be chosen such that the interactions between, for example, the said PDK1 and the interacting polypeptide comprising the amino acid sequence Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27), are substantially the same as between human PDK1 and a naturally occuring interacting polypeptide comprising the said amino acid sequence. It will be appreciated that the compound may bind to the PDK1, or may bind to the interacting polypeptide comprising the said amino acid sequence, for example a polypeptide derivable from PRK2.

A further aspect of the invention is a compound identifiable or identified by the said screening method. It will be appreciated that such a compound may be a modulator, for example an inhibitor of the PDK1 or PDK2 protein kinase activity of the PDK1 used in the screen and that the intention of the screen may be to identify compounds that act as modulators, for example inhibitors of the PDK1 or PDK2 protein kinase activity, even if the screen makes use of a binding assay rather than an enzymic activity assay. It will be appreciated that the modulatory, for example inhibitory action of a compound found to bind the protein kinase may be confirmed by performing an assay of enzymic activity (ie PDK1 and/or PDK2 protein kinase activity) in the presence of the compound.

A still further aspect of the invention is a compound (or polypeptide or polynucleotide) of the invention for use in medicine. A still further aspect of the invention is a polypeptide which comprises the amino acid sequence Phe/Tyr-Xaa-Xaa-Phe/Tyr-Zaa-Phe/Tyr (SEQ ID NO:25), for example Phe/Tyr-Xaa-Xaa-Phe/Tyr-Asp/Glu-Phe/Tyr (SEQ ID NO:26) or Phe/Tyr-Xaa-Xaa-Phe/Tyr-PhosphoSer/PhosphoThr-Phe/Tyr (SEQ ID NO:27), or a polypeptide consisting essentially of residues 51 to 404 of PDK1 or a fusion of a polypeptide consisting essentially of residues 51 to 404 of PDK1 wherein Zaa represents a negatively charged amino acid residue for use in medicine. Relevant preferences, for example concerning length, for the said polypeptides are as described above for polypeptides of the invention.

The compound (or polypeptide or polynucleotide) may be administered in any suitable way, usually parenterally, for example intravenously, intraperitoneally or intravesically, in standard sterile, non-pyrogenic formulations of diluents and carriers. The compound (or polypeptide or polynucleotide) may also be administered topically, which may be of particular benefit for treatment of surface wounds. The compound (or polypeptide or polynucleotide) may also be administered in a localised manner, for example by injection.

A further aspect of the invention is the use of a compound (or polypeptide or polynucleotide) as defined above in the manufacture of a medicament for the treatment of a patient in need of modulation of the insulin signalling pathway and/or signalling by PDK1, PDK2 or PRK2.

A compound that is capable of reducing the activity (ie the PDK1 and/or the PDK2 activity) of PDK1 may be useful in treating cancer. PDK1, for example via PKB and/or SGK, may be capable of providing a survival signal that protects cells from apoptosis induced in a variety of ways (reviewed in [8, 13]). Thus, such compounds may aid apoptosis. Reduction of the activity of PDK1 may promote apoptosis and may therefore be useful in treating cancer. Conditions in which aiding apoptosis may be of benefit may also include resolution of inflammation.

A compound is capable of increasing the activity of PDK1 may be useful in treating diabetes or obesity, or may be useful in inhibiting apoptosis. Increased activity of PDK1 may lead to increased levels of leptin, as discussed above, which may lead to weight loss; thus such compounds may lead to weight loss. For example, such compounds may suppress apoptosis, which may aid cell survival during or following cell damaging processes. It is believed that such compounds are useful in treating disease in which apoptosis is involved. Examples of such diseases include, but are not limited to, mechanical (including heat) tissue injury or ischaemic disease, for example stroke and myocardial infarction, neural injury and myocardial infarction. Thus the patient in need of modulation of the activity of PDK1 may be a patient with cancer or with diabetes, or a patient in need of inhibition of apoptosis, for example a patient suffering from tissue injury or ischaemic injury, including stroke.

Thus, a further aspect of the invention provides a method of treating a patient with an ischaemic disease the method comprising administering to the patient an effective amount of a compound identifiable by the screening methods of the invention.

A still further invention provides a use of a compound identifiable by the screening methods of the invention in the manufacture of a medicament for treating an ischaemic disease in a patient.

Thus, a further aspect of the invention provides a method of treating a patient with an ischaemic disease the method comprising administering to the patient an effective amount of a compound identifiable by the screening methods of the invention.

If the patient is a patient in need of promotion of apoptosis, for example a patient with cancer, it is preferred that the compound of the invention that is used in the preparation of the medicament is capable of reducing the activity of PDK1. If the patient is a patient with diabetes or a patient in need of inhibition of apoptosis, for example a patient with ischaemic disease, it is preferred that the compound of the invention that is used in the preparation of the medicament is capable of increasing the activity of SGK.

The invention will now be described by reference to the following Examples and Figures:

FIG. 1. Two hybrid interaction of PIF with the kinase domain of PDK1. Yeast were transformed with a pGAD10 plasmid (LEU2) harbouring the C-terminal 77 amino acids of PRK2 (termed PIF) in the presence of either a pAS2-1 plasmid (TRP1) coding for the expression of the wild type PDK1, the kinase domain of PDK1 (residues 1 to 404) or the PH domain of PDK1 (residues 428 to 556) or the empty pAS2-1 vector as a control (denoted by—(A). The position on the plate of each yeast strain is indicated in (A). In (B) the yeast are grown on SD-Leu and Trp deficient media in which all the strains of yeast are capable of growing independently of whether PIF is interacting with PDK1. An interaction between PIF and PDK1 leads to the expression of reporter genes coding for URA3 and HIS1 that will enable growth on SD-Leu-Trp-Ura-His+3-AT medium (C) as well as the induction of β-galactosidase gene expression (D). Alignment of the amino acid sequence of the C-terminal 77 amino acids of PRK2 with the equivalent region of AGC subfamily kinases indicated (E). Identical residues are denoted by white-on-black letters, and similar residues by grey boxes. PRK2 (SEQ ID NO:14; PRK1 (SEQ ID NO:15); PKBα (SEQ ID NO:16); P70S6k (SEQ ID NO:17); SGK (SEQ ID NO:18); PKCζ (SEQ ID NO:19); PKCα (SEQ ID NO:20); PKCβ (SEQ ID NO:21).

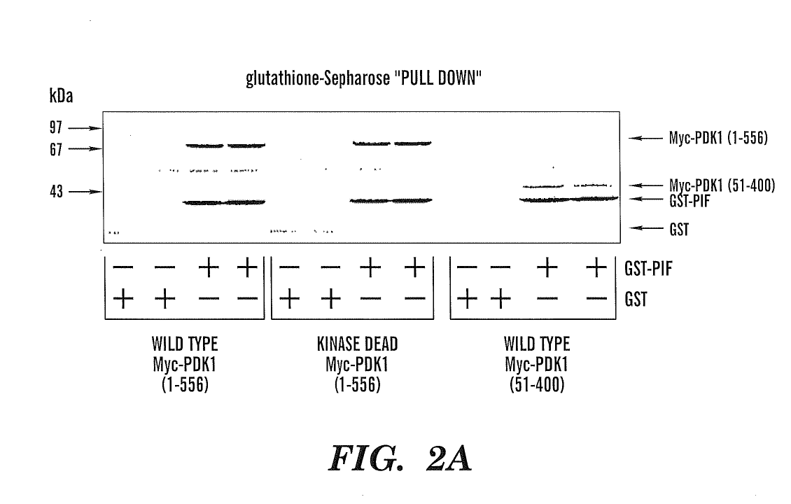

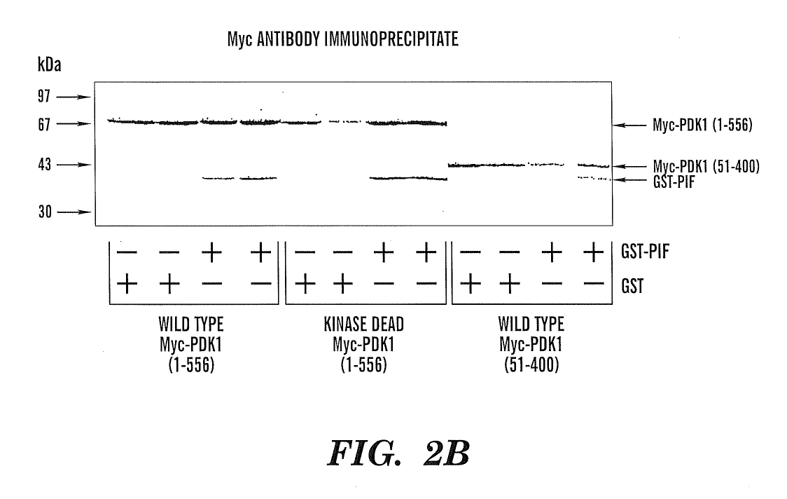

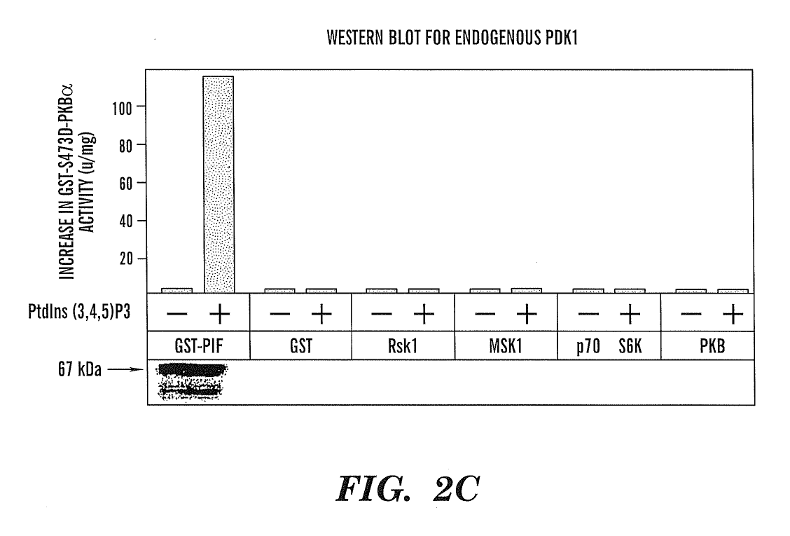

FIG. 2. Specific interaction of GST-PIF with PDK1 in 293 cells. (A+B) 293 cells were transiently transfected with DNA constructs expressing GST or GST-PIF together with either wild type Myc-PDK1, kinase dead Myc-PDK1 (K111A, D223A) or the kinase domain of Myc-PDK1 (residues 51 to 404). 36 h post transfection the cells were lysed and either the GST or GST-PIF purified from 350 μg of lysate by affinity chromatography of glutathione-Sepharose beads (A) or the Myc-PDK1 proteins immunoprecipitated using Myc antibodies covalently attached to Protein G-Sepharose (B). The total protein from each purification was electrophoresed on a 10% SDS/polyacrylamide gel and stained with Coomassie blue. The position of the molecular mass markers, glycogen phosphorylase (97 kDa), bovine serum albumin (67 kDa) and ovalbumin (43 kDa) are indicated. (C) 293 cells were transiently transfected with DNA constructs expressing either GST-PIF, GST alone, GST-p90RSK1, GST-MSK1, GST-p70 S6 kinase lacking the C-terminal 104 residues, and GST-PKBα. 36 hours post transfection the cells were lysed and the GST fusion proteins purified by affinity chromatography on glutathione-Sepharose beads. Each GST-fusion protein was incubated for 30 min at 30° C. with GST-S473D-PKBα and MgATP in the presence or absence of phospholipid vesicles containing 100 μM PtdCho, 100 μM PtdSer, 10 μM sn-1-stearoyl-2-arachidonoyl-D-PtdIns(3,4,5)P₃, and the increase in specific activity of GST-S473D-PKBα was determined relative to a control incubation in which the GST-S473D-PKBα fusion protein was omitted (average for 6 determinations, three independent experiments). The basal activity of GST-S473D-PKBα was 3.5 U/mg. 2 μg of each protein was electrophoresed on a 10% SDS/polyacrylamide gel and immunoblotted using a PDK1 antibody raised against the PDK1 protein (used at 0.2 μg/ml) to detect any endogenous PDK1 associated with the glutathione-Sepharose pull downs.

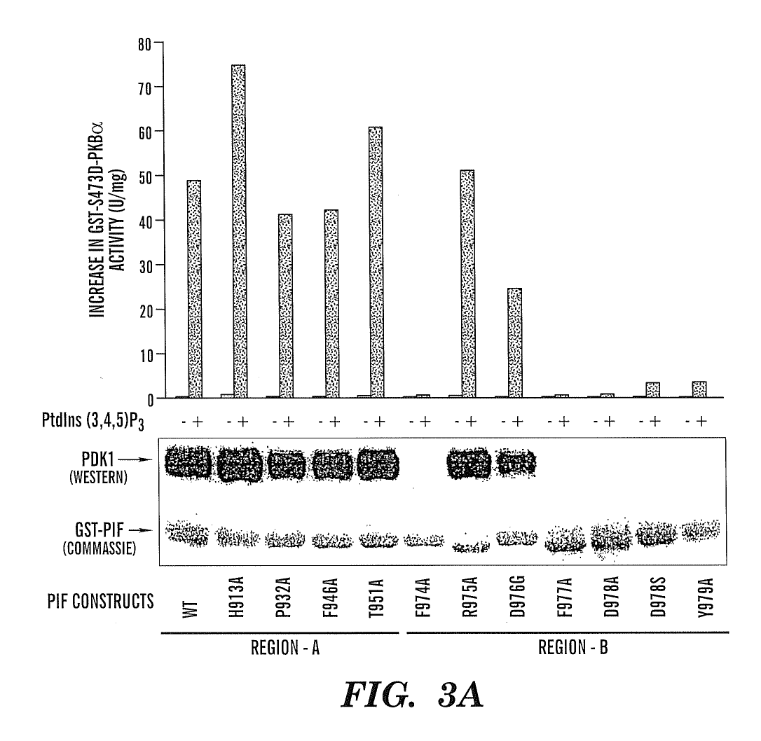

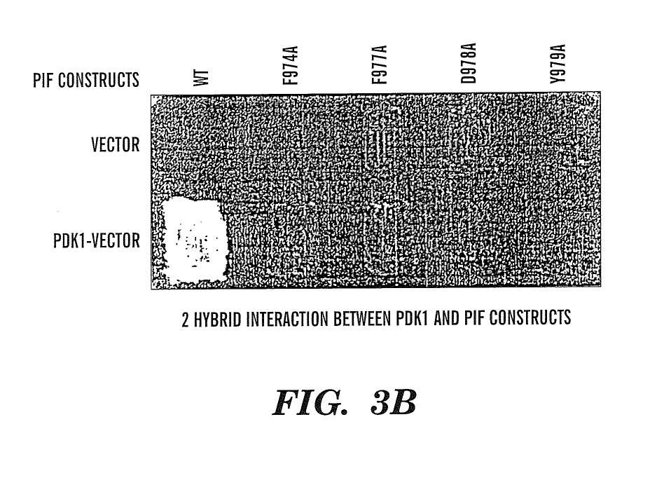

FIG. 3. Mapping the residues on PIF that are required for the interaction with PDK1. (A) 293 cells were transiently transfected with DNA constructs expressing either the wild type GST-PIF or the indicated point mutations of this protein. The cells were lysed and the GST fusion proteins purified by affinity chromatography on glutathione-Sepharose beads. Each GST-fusion protein was incubated for 30 min at 30° C. with GST-S473D-PKBα and MgATP in the presence or absence of phospholipid vesicles containing 100 μM PtdCho, 100 μM PtdSer, 10 μM sn-1-stearoyl-2-arachidonoyl-D-PtdIns(3,4,5)P₃, and the increase in specific activity of GST-S473D-PKBα was determined relative to a control incubation in which the GST fusion protein was omitted. The basal activity of GST-S473D-PKBα was 3.5 U/mg. 2 μg of each protein was electrophoresed on a 10% SDS/polyacrylamide gel and either immunoblotted using a PDK1 antibody raised against whole protein to detect any endogenous PDK1, or stained with Coomassie blue to establish the level of expression of the GST-PIF protein. Similar results were obtained in 5 separate experiments using 2 or more preparations of the wild type and mutant GST-PIF proteins. (B) Yeast harbouring the pAS2-1 wild type PDK1 vector (“PDK1-Vector”) or the empty pAS2-1 vector (“Vector”) were transformed with pGAD10 plasmid coding for the expression of the wild type PIF or the indicated mutants of PIF. The resulting yeast strain was grown on SD-Leu-Trp-Ura-His+3-AT medium which only permits growth of yeast constructs in which GST-PIF and PDK1 are capable of interacting.

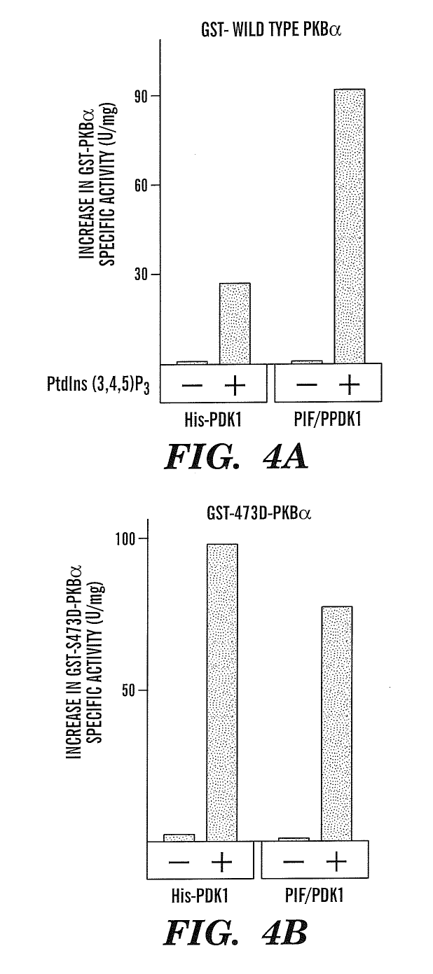

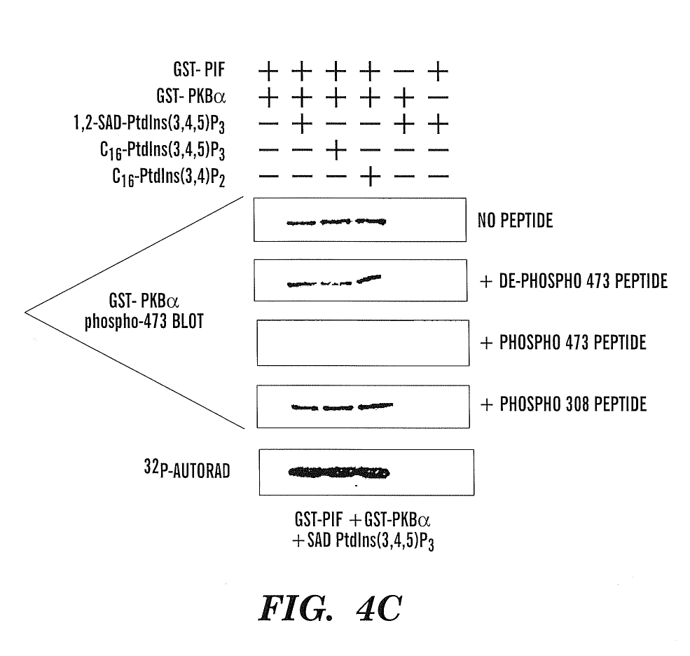

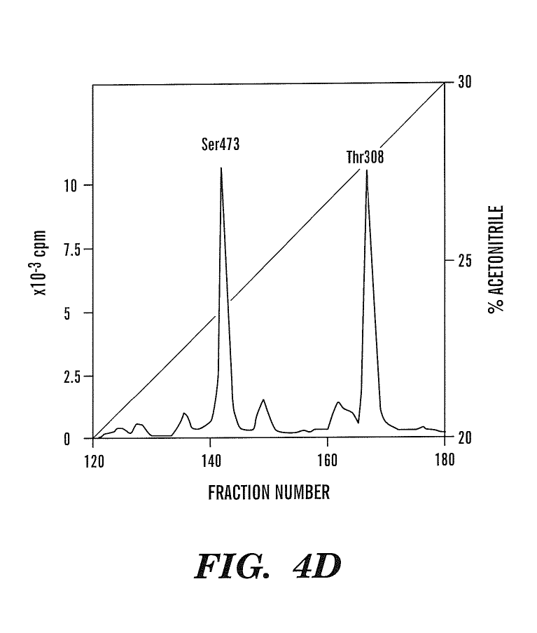

FIG. 4. Incubation of PIF with PKBα induces phosphorylation of PKBα at both Thr308 and Ser473. GST-PKBα (A) or GST-S473D-PKBα (B) was incubated at 30° C. for 1 h with MgATP in the presence of either His-PDK1 (0.1 μg) or GST-PIF purified from 293 cells which is associated with endogenous PDK1 (1 μg termed GST-PIF/PDK1) in the presence or absence of phospholipid vesicles containing 100 μM PtdCho, 100 μM PtdSer, and 10 μM sn-1-stearoyl-2-arachidonoyl-D-PtdIns(3,4,5)P₃. The increase in specific activity of GST-PKBα was determined, relative to a control incubation in which His-PDK1 or GST-PIF was omitted (average for 6 determinations, two independent experiments). The basal activity of GST-PKBα was 1.0 U/mg, GST-S473D-PKBα was 3.5 U/mg. Under the conditions used, a maximal activation of GST-PKBα (A) and GST-S473D-PKBα (B) was observed which could not be increased by prolonging the incubation period or increasing the concentration of His-PDK1 or GST-PIF added to the assay. (C) GST-PKBα was incubated with GST-PIF/PDK1 (1.0 μg) as in (A). The reactions were terminated by making the solutions 1% in SDS, the samples were subjected to SDS/polyacylamide gel electrophoresis and immunoblotted using the P-Ser473 antibody that was incubated in the presence or absence of the following synthetic peptides (all at 10 μg/ml) corresponding to residues 467 to 477 of PKBα phosphorylated at the residue equivalent to Ser473 (phospho-473 peptide), the same peptide that was not phosphorylated (de-phospho-473 peptide), or a peptide corresponding to residues 301 to 313 of PKBα phosphorylated at the residue equivalent to Thr308 (phospho-308 peptide). (D) as in (A) except radioactive Mg[γ³²P]ATP (10⁶ cpm per nmol) was employed, the samples alkylated with 4-vinylpyridine, electrophoresed on a 10% polyacrylamide gel. The phosphorylated GST-PKBα was excised from the gel, digested with trypsin and applied to a Vydac 218TP54 C₁₈ column (Separations Group, Hesperia, Calif.) equilibrated in 0.1% (v/v) trifluoroacetic acid (TFA), as described previously [17]. The column was developed with a linear acetonitrile gradient (diagonal line) at a flow rate of 0.8 ml/min and fractions of 0.4 ml were collected. Between 50-70% of the radioactivity applied to the column was recovered in the major ³²P-containing peptides eluting at 24% and 26% acetonitrile, which were identified (see results) as the PKBα tryptic peptides phosphorylated at Ser473 and Thr308 respectively.

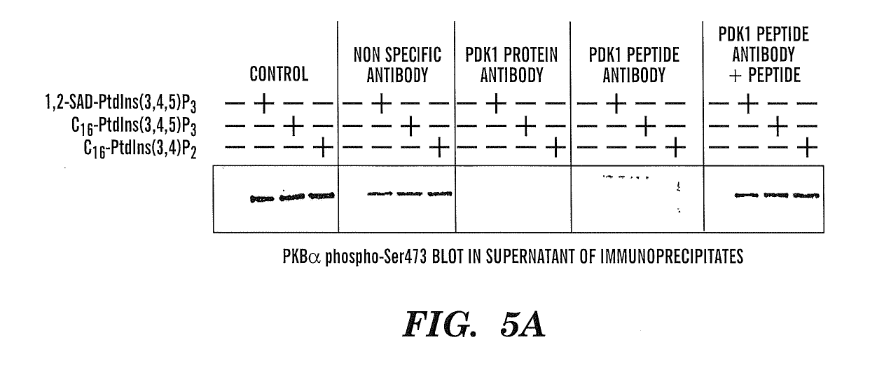

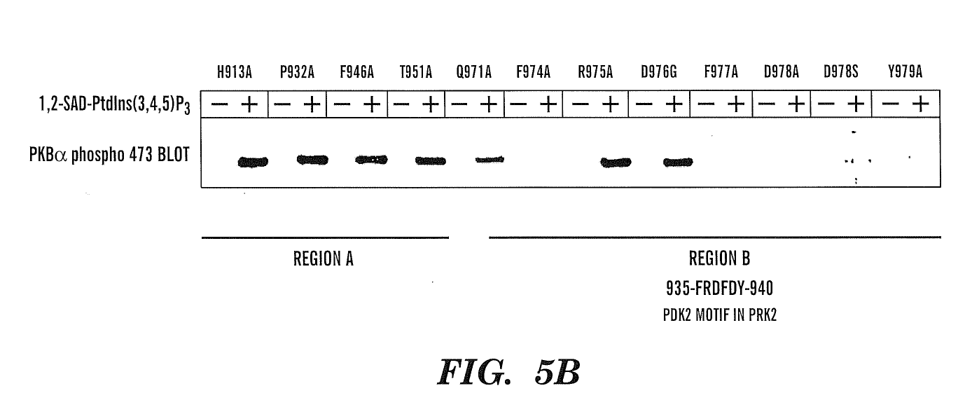

FIG. 5. The ability of GST-PIF to phosphorylate PKBα at Ser473 is dependent upon the presence of PDK1. GST-PIF purified from 293 cells, FIG. 2C (20 μg in 50 μl) was incubated with Protein G-Sepharose (10 μl) conjugated with either no antibody (control), MSK1 antibody A [40] (5 μg, non specific antibody), a PDK1 antibody raised against the whole protein (5 μg) or a PDK1 peptide antibody raised against the 16 C-terminal amino acid residues of PDK1 (5 μg, peptide antibody) in the presence or absence of the PDK1 C-terminal peptide immunogen (0.2 mM) to which this antibody was raised. After incubation on a shaking platform for 60 min at 4° C., the suspension was centrifuged and GST-PIF in the supernatant (2.0 μg) was incubated with GST-PKBα and MgATP for 60 min at 30° C. in the presence or absence of phospholipid vesicles containing 100 μM PtdCho, 100 μM PtdSer, and 10 μM sn-1-stearoyl-2-arachidonoyl-D-PtdIns(3,4,5)P₃ (1,2-SAD-PtdIns(3,4,5)P₃), or sn-1,2 di-palmitoyl-D-PtdIns(3,4,5)P₃ (C₁₆-PtdIns(3,4,5)P₃) or sn-1,2 di-palmitoyl-D-PtdIns(3,4)P₂ (C₁₆-PtdIns(3,4)P₂). The reactions were terminated by making the solutions 1% in SDS, and the samples were subjected to SDS/polyacylamide gel electrophoresis and immunoblotted using the P-Ser473 antibody. Identical results were obtained in 2 separate experiments (B) The wild type and mutant GST-PIF proteins (2.0 μg) used in FIG. 3 were incubated with GST-PKBα and MgATP for 60 min at 30° C. in the presence or absence of lipid vesicles containing 10 μM sn-1-stearoyl-2-arachidonoyl-D-PtdIns(3,4,5)P₃ and the degree of phosphorylation of PKBα at Ser473 was established by immunoblotting. Similar results were obtained in 4 separate experiments using 2 different preparations of the wild type and mutant GST-PIF proteins.

FIG. 6. Conversion of PDK1 into an enzyme that can phosphorylate both Thr308 and Ser473 of PKB by addition of a synthetic peptide. (A) His-PDK1 (20 ng) was incubated in the presence or absence of the following peptide (all at 20 M) “Region B peptide” (residues 969 to 984 of PIF), “D978A Region B peptide” (residues 919 to 945 of PIF in which Asp 978 is mutated to Ala) or “Region A peptide” (residues 927 to 951 of PIF). After allowing to stand for 1 h at 4° C. GST-PKBα, MgATP and the indicated phospholipid vesicles containing 100 μM PtdCho, 100 μM PtdSer, and either 10 μM sn-1-stearoyl-2-arachidonoyl-D-PtdIns(3,4,5)P₃ (1,2-SAD-PtdIns(3,4,5)P₃), or sn-1,2 di-palmitoyl-D-PtdIns(3,4,5)P₃ (C₁₆-PtdIns(3,4,5)P₃) or sn-1,2 di-palmitoyl-D-PtdIns(3,4)P₂ (C₁₆-PtdIns(3,4)P₂) were added. After 1 h at 30° C., the reactions were terminated by making the solutions 1% in SDS, the samples were subjected to SDS/polyacylamide gel electrophoresis and immunoblotted using P-Ser473 antibody. Identical results were obtained in 2 separate experiments. (B to D) His-PDK1 was incubated for 1 h on ice as above in the presence of “Region B peptide” (B), “D978A-Region B peptide” (C) or no peptide (D) and then incubated with Mg[γ³²P]ATP and phospholipid vesicles containing 100 μM PtdCho, 100 μM PtdSer, and 10 μM sn-1-stearoyl-2-arachidonoyl-D-PtdIns(3,4,5)P₃. After 1 h at 30° C. the reactions were terminated and the residues on PKB that became phosphorylated under these conditions were established as described in the legend to FIG. 4.

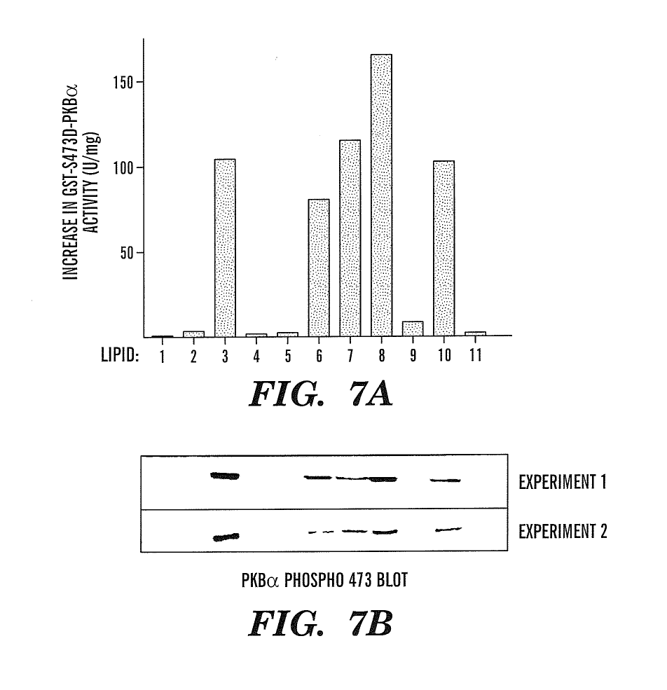

FIG. 7. Specificity of phosphoinositide-dependent PIF/PDK1-induced phosphorylation of Thr308 and Ser473 of PKBα. GST-PKBα was incubated for 60 min at 30° C. with GST-PIF purified from 293 cells (which is associated with endogenous PDK1 see FIG. 3) (1.0 μg), MgATP and phospholipid vesicles containing 100 μM PtdCho, 100 μM PtdSer, and various PtdIns lipids (numbered 1-11, see below) all at a final concentration of 10 μM in the assay. In panel A the reactions were terminated by the addition of Triton X100 and the increase in specific activity of GST-S473D-PKBα (U/mg), determined, relative to a control incubation in which GST-PIF was omitted (±SEM for 6 determinations, two independent experiments). The basal activity of GST-S473D-PKBα was 3.5 U/mg. In Panel B the reactions were terminated by making the solutions 1% in SDS, the samples were subjected to SDS/polyacylamide gel electrophoresis and the extent of PKBα phosphorylation was established using the phospho-specific antibody that recognises PKBα phosphorylated at Ser 473. In B the results of 2 separate experiments with different preparations of lipid are shown. Lipid 1, Buffer control; Lipid 2, PtdIns 3P; Lipid 3, n-1,2-dipalmitoyl PtdIns(3,4)P₂; Lipid 4, PtdIns(3,5)P₂; Lipid 5, PtdIns(4,5)P₂; Lipid 6, sn-1,2-dipalmitoyl D-PtdIns(3,4,5)P₃; Lipid 7, rac-1,2-dilinoleoyl phosphatidyl D/L-myo-inositol 3,4,5trisphosphate; Lipid 8, sn-1-stearoyl, 2-arachidonoyl D-PtdIns(3,4,5,)P₃; Lipid 9, sn-1-stearoyl, 2-arachidonoyl L-PtdIns(3,4,5,)P₃; Lipid 10, sn-2-stearoyl, 3-arachidonoyl D-PtdIns(3,4,5,)P₃; Lipid 11, sn-2-ste aroyl, 3-arachidonoyl L-PtdIns(3,4,5,)P₃.

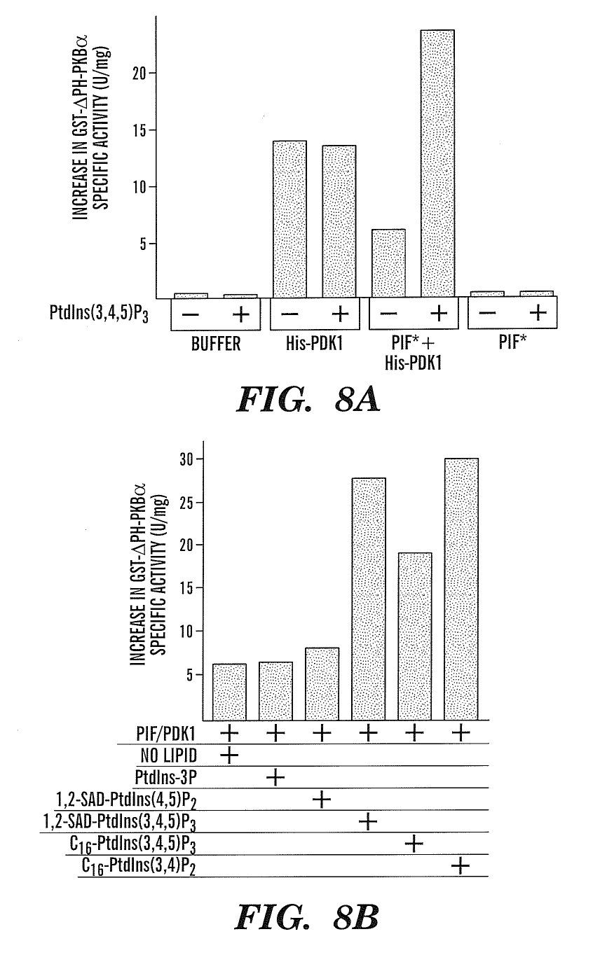

FIG. 8. Maximal activation of PKB by PDK1/PIF requires PtdIns(3,4,5)P₃ to interact with both PKB and PDK1. GST-ΔPH-PKBα was incubated at 30° C. for 1 h with MgATP in the presence of either buffer (control), His-PDK1 (0.1 μg), or His-PDK1 (0.1 μg)+GST-PIF*(1.0 μg in which all the PDK1 had been removed by immuno-depletion), or GST-PIF* alone (1.0 μg) or GST-PIF purified from 293 cells which is associated with endogenous PDK1 (2 μg termed GST-PIF/PDK1) in the presence or absence of phospholipid vesicles containing 100 μM PtdCho, 100 μM PtdSer, and 10 μM sn-1-ste aroyl-2-arachidonoyl-D-PtdIns(3,4,5)P₃. The increase in specific activity of GST-ΔPH-PKBα was determined, relative to a control incubation in which His-PDK1 or GST-PIF was omitted (average for three determinations in one experiment are shown, similar results were obtained in three other separate experiments). The basal activity of GST-ΔPH-PKBα was 3.0 U/mg.

FIG. 9. Fractionation of rat brain extract. (A) Brain extract was fractionated by batchwise chromatography on Q-Sepharose followed by gradient elution from Heparin-Sepharose (see methods). The column fractions were assayed for their ability to phosphorylate PKBα at Thr308 (measured by the increase in GST-S473D-PKBα activity) and Ser473 (using the P-473 antibody) in the presence (open squares) or absence (closed squares) of 10 μM 1,2-SAD-PtdIns(3,4,5)P₃. The broken line indicates the salt gradient. (b) Frations 7 and 8 of the Heparin-Sepharose column were pooled and aliquots (0.1 ml) incubated with Protein G_Sepharose (10 μl) conjugated to an MSK1 antibody A [41] (5 μg, non-specific antibody), a PDK1 antibody raised against the whole protein (5 μg), or no antibody (control). After incubation on a shaking platform for 60 min at 4° C., the suspensionwas centrifuged, and an aliquot of the supernatant (5 μl) incubated for 60 min at 30° C. ith either GST-S473D-PKBα (PDK1 assay) or GST-PKBα (PDK2 assay) and MgATP in the presence or absence of 10 μM 1,2-SAD-PtdIns(3,4,5)P₃ or C₆-PtdIns(3,4)P₂. The phosphorylation of PKBα at Ser473 and Thr308 was then measured as in A. Similar results were obtained in experiments from two separate purifications.

FIG. 10. PDK1 sequence (SEQ ID NO:31)

FIG. 11. PRK2 sequence (SEQ ID NO:32)

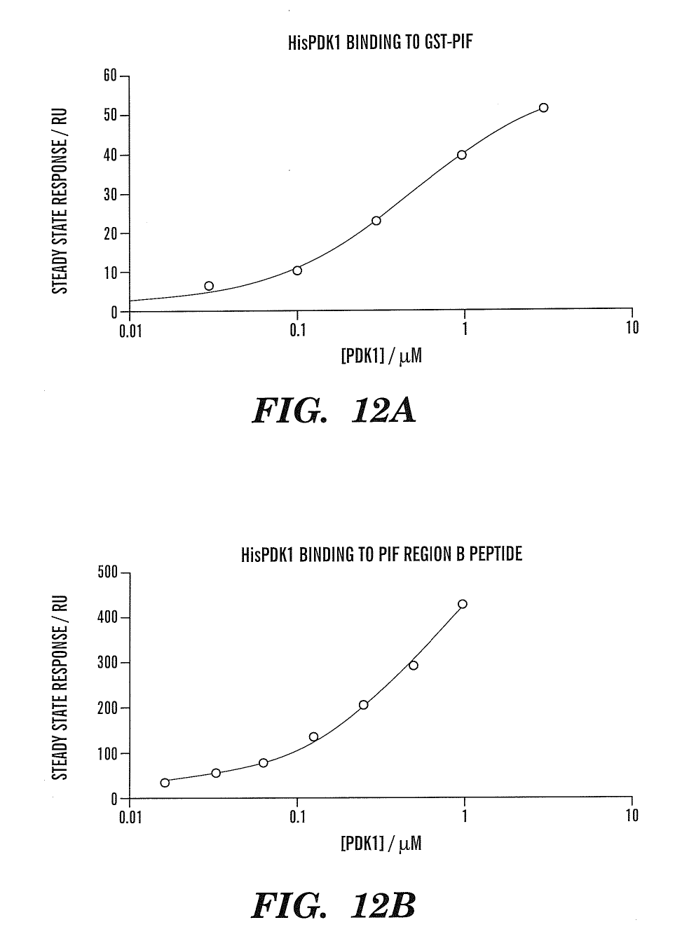

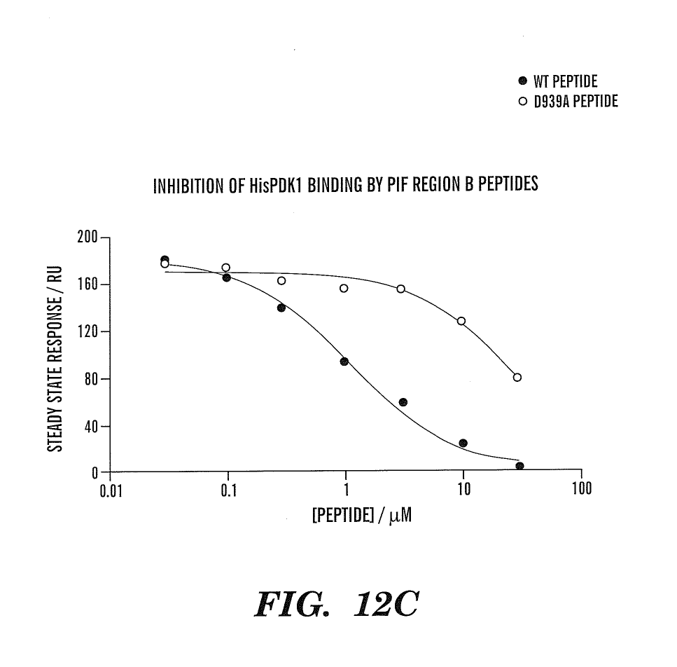

FIG. 12. Quantitative analysis of the binding of PDK1 to PIF. Surface Plasmon Resonance measurements were carried out on a BiaCore instrument as described in the methods section. His-PDK1 was injected at the indicated concentrations over (A) 2000Rus of GST-PIF, which was immobilised by amine coupling to a CM5 Sensorchip, or (B) 740 Rus of biotinylated wild type PIF, which was immobilised on a Sensorchip SA. The responses at steady state binding were recorded. (C) shows the inhibition of the steady-state response when 500 nM H is PDK1 was co-injected with the indicated concentrations of the wild type and D393A peptides. All data are single determinations from a representative experiment that was repeated at least 3 times with similar results.

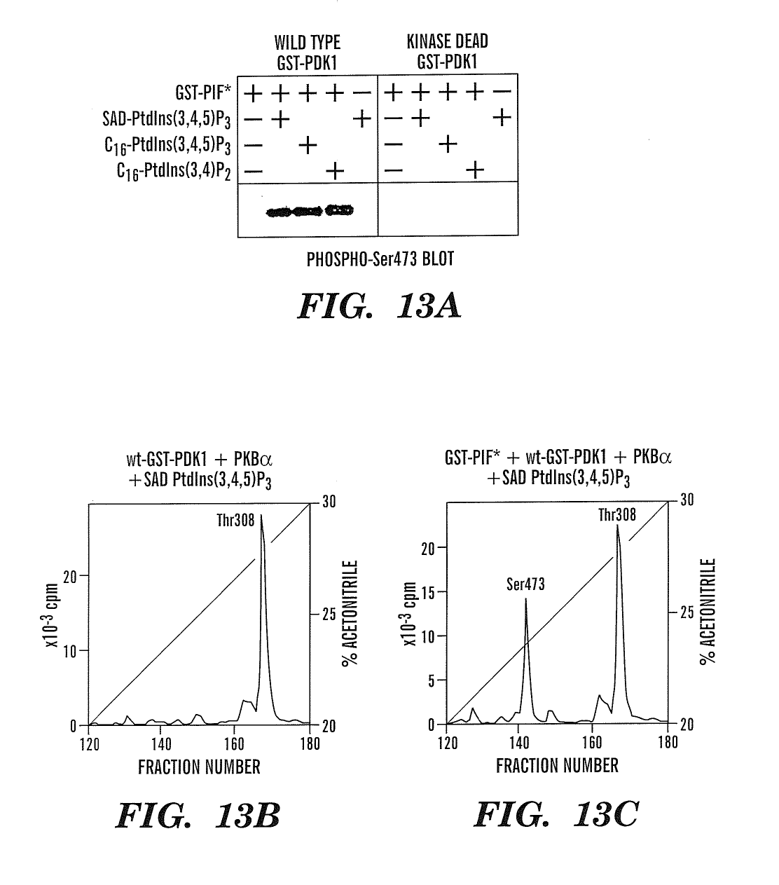

FIG. 13. Conversion of PDK1 into an enzyme that can phosphorylate both Thr308 and Ser473 of PKB by addition of PIF. (A) GST-PIF in which all the PDK1 has been removed by immuno-depletion as described in FIG. 5 (1.0 μg, termed GST-PIF*) or the equivalent volume of Buffer was added to 20 ng of wild type GST-PDK1 or kinase dead-GST-PDK1 (D223A mutant [18]). After allowing to stand for 1 h at 4° C. GST-PKBα, MgATP and the indicated phospholipid vesicles containing 100 μM PtdCho, 100 μM PtdSer, and either 10 μM sn-1-stearoyl-2-arachidonoyl-D-PtdIns(3,4,5)P₃ (1,2-SAD-PtdIns(3,4,5)P₃), or sn-1,2 di-palmitoyl-D-PtdIns(3,4,5)P₃ (C₁₆-PtdIns(3,4,5)P₃) or sn-1,2 di-palmitoyl-D-PtdIns(3,4)P₂ (C₁₆-PtdIns(3,4)P₂) were added. After 1 h at 30° C., the reactions were terminated by making the solutions 1% in SDS, the samples were subjected to SDS/polyacylamide gel electrophoresis and immunoblotted using the P-473 antibody. Identical results were obtained in 2 separate experiments. (B and C) wild type GST-PDK1 (20 ng) was incubated in the absence (B) or presence of GST-PIF* for 1 h on ice and then incubated with Mg[Y³²P]ATP and phospholipid vesicles containing 100 μM PtdCho, 100 μM PtdSer, and either 10 μM sn-1-stearoyl-2-arachidonoyl-D-PtdIns(3,4,5)P₃. After 1 h at 30° C. the reactions were terminated and the residues on PKB that became phosphorylated under these conditions were determined as described in the legend to FIG. 5.

FIG. 14: PRK1 sequence (SEQ ID NO:33)

FIG. 15: PKCζ sequence (SEQ ID NO:34)

### EXAMPLE 1

### PDK1 and PDK2 May be the Same Enzyme; PDK1 Displays PDK2 Activity in the Presence of a Synthetic Peptide

Abbreviations: PKB, Protein kinase B; PtdIns, Phosphatidylinositol; PI 3-kinase, Phosphoinositide 3-kinase; PtdCho, Phosphatidylcholine; PtdSer, Phosphatidylserine; PH, pleckstrin homology; RSK, Ribosomal S6 kinase; MSK, Mitogen and Stress Stimulated kinase.

Results. Using the yeast two hybrid system we observed that the kinase domain of PDK1 interacted with a region of the Protein Kinase C-Related Kinase-2 (PRK2), termed the PDK1 Interacting Fragment (PIF). PIF is situated C-terminal to the kinase domain of PRK2, and contains a consensus motif for phosphorylation by PDK2, similar to that found in PKBα, except that the residue equivalent to Ser473 is changed to Asp. Mutation of any of the conserved aromatic residues in the PDK2 motif of PIF or mutation of the Asp residue to either Ala or Ser prevented the interaction of PIF with PDK1. Remarkably, the interaction of PDK1 with PIF or a 24 residue synthetic peptide whose sequence encompasses the PDK2 consensus sequence of PIF, converted PDK1 from an enzyme that could only phosphorylate Thr308 of PKBα to a kinase that phosphorylates both Thr308 and Ser473 of PKBα in a PtdIns(3,4,5)P₃ dependent manner. Furthermore, the interaction of PIF with PDK1, converted the latter from a form which is not activated by PtdIns(3,4,5)P₃ directly to a form which is activated 3 to 4-fold by PtdIns(3,4,5)P₃ in vitro. We have partially purified a kinase from brain extract that phosphorylates Ser 473 of PKBα in a PtdIns(3,4,5)P₃ dependent manner which is immunoprecipitated with a PDK1 antibody.

Conclusions. PDK1 possesses the intrinsic ability to phosphorylate Ser473 as well as Thr308 of PKBα in a 3-phosphoinositide dependent manner when complexed through its kinase domain to PIF. These finding raise the possibility that PDK1 and PDK2 are the same enzyme, and that the substrate specificity and activity of PDK1 is regulated through its interaction with another protein(s) in vivo. PRK2 may be a substrate for PDK1.

The C-terminal region of PRK2 interacts specifically with PDK1. A yeast two-hybrid screen was carried out to identify proteins expressed in human skeletal muscle that interact with PDK1. We identified one clone corresponding to the C-terminal 77 amino acids of PRK2 that yielded a positive interaction with full length PDK1 as well as with the isolated kinase domain (residues 51-404), but not with the PH domain of PDK1 (FIG. 1A to 1D). We have termed this region of PRK2 the “PDK1-Interacting Fragment” (PIF). PRK2 is a Rho and lipid dependent protein kinase [27,28] that, like PDK1 and PKB belongs to the AGC subfamily of protein kinases. The PIF region in PRK2 lies immediately C-terminal to the kinase catalytic domain, in a region where there is high sequence homology between AGC subfamily kinases (FIG. 1E). Most notably the C-terminus of PIF contains the consensus sequence for PDK2 phosphorylation (Phe/Tyr-Xaa-Xaa-Phe/Tyr-Ser/Thr-Phe/Tyr) (SEQ ID NO:30), except that in PRK2 the Ser/Thr residue equivalent to Ser473 of PKB is replaced with a negatively charged Asp residue (Asp978), perhaps mimicking a phosphorylated state.

In order to confirm that PDK1 did indeed interact with PIF, we expressed PIF as a glutathione-5-transferase (GST) fusion protein in 293 cells together with Myc epitope-tagged PDK1. The cells were lysed and the GST-PIF affinity purified by chromatography on glutathione-Sepharose (FIG. 2A) or the Myc-PDK1 isolated by immunoprecipitation using a Myc antibody (FIG. 2B). The samples were made 1% in SDS, then electrophoresed on an SDS-polyacrylamide gel and the proteins visualised by staining with Coomassie blue (FIG. 2). A near-stoichiometric complex was observed between GST-PIF and wild type PDK1, with the isolated kinase of PDK1 or with a catalytically-inactive mutant. The complex formed between PDK1 and GST-PIF could not be dissociated at concentrations of LiBr up to 2 M (a strong chaotrophic agent) or by incubation with 1% (by vol) Triton X100 (data not shown), indicating a very strong interaction. No interaction was observed between either GST and PDK1 (FIG. 2) or between GST-PIF and epitope tagged PKB, p70 S6 kinase, or 6-phosphofructo-2-kinase when each of these were coexpressed with PIF in 293 cells (data not shown). Analysis of the interaction using Surface Plasmon Resonance confirmed that GST-PIF was capable of associating directly with purified His-tagged PDK1 with an estimated equilibrium dissociation constant of 600 nM (FIG. 12). No association between GST-PIF and His-PKBα was detected using this method.

PIF binds specifically to the endogenous PDK1 in 293 cells. GST-PIF purified from 293 cells was found to be associated with the endogenous PDK1 as judged by Western blotting. In 293 cells, two PDK1 immunoreactive bands are observed running at 63 kDa and 66 kDa, and both were observed to co-purify with GST-PIF (FIG. 2C). The PDK1 associated with GST-PIF (0.5 μg) was active because in the presence of MgATP and PtdIns(3,4,5) P₃ it greatly activated a GST-A473D-PKBα mutant (FIG. 2C). Thus, PDK1 bound to PIF can phosphorylate PKB at Thr308 and the interaction between PIF and the kinase domain of PDK1 does not interfere with the activation of PKB. In contrast PKB, p70 S6 kinase, p90 RSK1 or MSK1, when expressed in 293 cells as GST fusion proteins, were not associated with any endogenous PDK1 after their precipitation on glutathione-Sepharose, as judged by immunoblotting and failure to activate GST-S473DPKBα in the presence of MgATP and PtdIns(3,4,5)P₃ (FIG. 2C).

The “PDK2 substrate motif” mediates the interaction between PIF and PDK1. We have termed the N-terminal 53 amino acids of PIF “Region A” and the C-terminal 24 amino acids “Region B” (FIG. 1E). In order to identify the amino acid residues in PIF that were required for the interaction of this fragment with the kinase domain of PDK1, we mutated many of the residues in PIF that are conserved between the AGC subfamily of protein kinases to Ala. The mutant GST-PIF proteins were expressed in 293 cells at similar levels, and the amount of endogenous PDK1 activity and PDK1 protein associated with each of the purified constructs was determined (FIG. 3A). Mutation of the conserved residues in Region A did not prevent the association of PDK1 with GST-PIF. Remarkably, however, the mutation to Ala of any of the conserved aromatic residues in the PDK2 phosphorylation site motif in Region B (F974, F977 and Y979) completely abolished the association of endogenous PDK1 with these GST-PIF mutants (FIG. 3A). Furthermore, mutation of Asp978 (the residue that is equivalent to Ser473 in the PKB phosphorylation site) to either Ala or Ser also abolished the association of PDK1 with PIF. In contrast, mutation of the residues located 2 and 3 amino acids N-terminal to Asp978 (e.g.Asp976, Arg975), which are not conserved between the AGC subfamily members) did not affect the association of PDK1 with PIF (FIG. 3A). A yeast 2 hybrid screen was also used to verify these findings (FIG. 3B).

A strong interaction (K_{d} 800 nM) between His-PDK1 and an immobilised and biotinylated 24 residue synthetic peptide corresponding to the “Region B peptide” was detected using Surface Plasmon Resonance measurements. This interaction was competed by the addition of free peptide, and analysis of this competition revealed that the K_{d} of this interaction was 1.5 μM. In contrast, a mutant peptide in which the residue corresponding to Asp978 was changed to Ala (termed “D978A-Region B peptide”) interacted with PDK1>fold 10-less strongly (FIG. 13). Incubation of purified GST-PIF expressed in 293 cells with the Region B peptide (100 μM) resulted in the dissociation of the endogenous PDK1 from GST-PIF, whereas a Region B peptide in which Asp978 was mutated to Ala was incapable of inducing the dissociation of PDK1 from PIF (data not shown).

GST-PIF converts PDK1 to a form that is able to phosphorylate Ser473 as well as Thr308 in PKBα. When the purified GST-PIF which is associated with the endogenous PDK1 of 293 cells (FIG. 2C) was incubated with GST-PKBα in the presence of MgATP and PtdIns(3,4,5)P₃ we noticed that GST-PKBα could be activated to a 3-fold higher maximum specific activity (˜90 U/mg) than is achievable by incubation of GST-PKBα with very high concentrations of His-PDK1 alone (˜30 U/mg, FIG. 4A). In contrast the mutant form of PKBα in which the Ser473 phosphorylation site is mutated to Asp (GST-S473D-PKBα) was activated to the same maximum specific activity by either PDK1 of PIF/PDK1 (FIG. 4B). These findings raised the possibility that GST-PIF/PDK1 had induced phosphorylation of PKBα at Ser473 as well as Thr308. In order to investigate this possibility we prepared phospho-specific antibodies that only recognise PKB that has been phosphorylated at Ser473 (termed P-473 antibody). This antibody did not recognise GST-PKBα that had been incubated with GST-PIF and MgATP in the absence of PtdIns(3,4,5)P₃ or PtdIns(3,4)P₂, nor did PKBα become phosphorylated under these conditions (FIG. 4C). Following the addition of PtdIns(3,4,5)P₃ or PtdIns(3,4)P₂, GST-PKBα became strongly phosphorylated and recognised by the P-473 antibody. Incubation of the P-473 antibody with the phosphorylated Ser473 peptide immunogen used to raise the antibody (but not with the dephosphorylated peptide or a phosphopeptide corresponding to the sequence surrounding Thr308) abolished its recognition of GST-PKBα (FIG. 4C). The phosphorylation at Ser473 observed in these experiments was dependent upon the presence of MgATP in the reaction and was abolished if the GST-PIF was heated for 2 min at 65° C. (data not shown). A much higher concentration of His-PDK1 that was not bound to PIF induced no phosphorylation of Ser473 in the presence or absence of PtdIns(3,4,5)P₃.

In order to identify all the residue(s) in PKBα phosphorylated by the GST-PIF/PDK1 complex the ³²P-labelled GST-PKBα was digested with trypsin and chromatographed on a C₁₈ column [14]. Two major ³²P-labelled peptides were observed that eluted at 24% and 26% acetonitrile (FIG. 4D). These peptides coeluted with the ³²P-labelled tryptic peptide containing Ser473 and Thr308, respectively [14, 18] and the identity of these peptides and the location of the phosphorylated residues (Thr308 and Ser473) was established by MALDI-TOF mass spectrometry and solid phase amino acid sequencing (data not shown). The peptide eluting at 24% acetonitrile contained phosphoserine, and when subjected to solid phase sequencing, ³²P-radioactivity was released after the eighth cycle of Edman degradation (data not shown). Its identity was established by MALDI-TOF mass spectrometry and revealed that the molecular weight of the peptide (1732.8) was identical to that expected for the tryptic phosphopeptide comprising residues 466-480 and phosphorylated at Ser473. The peptide eluting at 26% acetonitrile contained phosphothreonine and when subjected to solid phase sequencing, ³²P-radioactivity was released after the first cycle of Edman degradation (data not shown). MALDI-TOF mass spectrometry revealed its molecular weight (2573.3) was identical to that expected for the tryptic phosphopeptide comprising residues 308-328 and phosphorylated at Thr308 (calculated mass 2573.1).

The PtdIns(3,4,5)P₃ dependent phosphorylation of PKBα at Ser473 is mediated by PDK1. The results presented in FIG. 4 could be explained in two different ways. Firstly, the interaction of PIF with PDK1 might have allowed the latter to phosphorylate Ser473 as well as Thr308. Secondly, purified GST-PIF might be complexed to two different protein kinases, namely PDK1 and PDK2. In order to distinguish between these possibilities we immuno-depleted the PDK1 from GST-PIF using 2 different antibodies. We then tested the ability of the supernatant to phosphorylate PKBα at Ser473. Strikingly, both PDK1 antibodies completely removed all the Ser473 kinase activity associated with GST-PIF (FIG. 5A) as well as the Thr308 kinase activity (data not shown). A peptide immunogen to which one of the antibodies was raised prevented immuno-depletion of the PDK2 activity associated with PIF (FIG. 5A).

These results indicated that the PtdIns(3,4,5)P₃ dependent Ser473 kinase activity associated with GST-PIF was catalysed by PDK1 rather than a separate enzyme. Consistent with this notion, all the mutant GST-PIF proteins purified from 293 cells that were able to interact with the endogenous PDK1 (FIG. 3) were able to induce the phosphorylation of PKBα at Ser473 (FIG. 5B). In contrast, the point mutations in GST-PIF that abolish its association with PDK1 also prevented the phosphorylation of PKBα at Ser473 (FIG. 5B).

Conversion of PDK1 into a kinase that phosphorylates Thr308 and Ser473 of PKBα. It could still be argued that the phosphorylation of Ser473 was catalysed by a protein kinase distinct from PDK1, but that this kinase was tightly complexed to PDK1 and not dissociated by chaotrophic reagents, detergents and therefore also removed from GST-PIF by immuno-depletion of PDK1. In order to address this point we removed PDK1 from purified GST-PIF by immuno-depletion and added the PDK1-depleted PIF (termed GST-PIF*) to purified wild type GST-PDK1 (FIG. 14) or His-PDK1 (data not shown). Initial rate studies demonstrated that, under the conditions used, the GST-PIF*/PDK1 complex phosphorylates Thr308 of PKBα at twice the rate of Ser473 (data not shown). Moreover, the ability of GST-PIF* to convert PDK1 into a Ser473 PKBα kinase was dependent upon PDK1 catalytic activity, since a catalytically inactive GST-PDK1 mutant was not capable of phosphorylating PKBα at Ser473 in the presence of GST-PIF* (FIG. 13).

The above experiments do not exclude the possibility that another protein associated with PIF (rather than PIF itself) is capable of interacting with PDK1 and altering its substrate specificity. This possibility was eliminated by the demonstration that a 24 residue synthetic peptide corresponding to “Region B” of PIF was capable of inducing His-PDK1 to phosphorylate PKBα at Ser473 in a 3-phosphoinositide dependent manner (FIG. 6). In contrast, a Region B peptide in which Asp978 was mutated to Ala was far less effective at inducing this alteration in the specificity of PDK1. The region B peptide converts the wild type GST-PDK1, but not the kinase dead GST-PDK1, into a kinase which can phosphorylate Ser473 (data not shown).

Lipid specificity of Ser473 phosphorylation. We compared the ability of a panel of PtdIns derivatives to stimulate the phosphorylation of Thr308 and Ser473 by the GST-PIF/PDK1 complex in the presence of a vesicle background containing PtdCho/PtdSer [17]. Synthetic sn-1-ste aroyl, 2-arachidonoyl D-PtdIns(3,4,5,)P₃ (1,2-SAD-PtdIns(3,4,5)P₃ the predominant form of PtdIns(3,4,5)P₃ which occurs naturally, lipid 8 in FIG. 7) and synthetic 2,3-SAD-PtdIns(3,4,5,)P₃ (lipid 10 in FIG. 7) were highly effective in inducing both the phosphorylation of GST-PKBα at Ser473 (FIG. 7B) and the phosphorylation of Thr308 (measured by the activation of GST-S473D-PKBα), indicating that the enantiomeric configuration of the glycerol moiety is not an important determinant of specificity. The L-enantiomers of these lipids (Lipids 9 and 11, FIG. 7) failed to induce a significant phosphorylation of PKB at Ser473 or activation of GST-S473D-PKBα. rac-1,2-dilinoleoyl phosphatidyl D/L-myo-inositol 3,4,5-trisphosphate (linoleic acid is C18:2, Lipid 7 FIG. 7), sn-1,2-dipalmitoyl D-PtdIns(3,4,5)P₃, (Lipid 6, FIG. 7) as well as sn-1,2-dipalmitoyl PtdIns(3,4)P₂ (Lipid 3 FIG. 7) were also effective at inducing the PIF/PDK1 complex to phosphorylate PKBα at Ser473 and Thr308. However, PtdIns-3P (lipid 2, FIG. 7), PtdIns(3,5)P₂ (lipid 4 FIG. 7) and PtdIns(4,5)P₂ (lipid 5 FIG. 7), did not induce the PIF/PDK1 complex to phosphorylate PKBα at Ser473 or Thr308. In the absence of the PIF/PDK1 complex none of the PtdIns derivatives tested induced any phosphorylation of Ser473 or activation of GST-S473D-PKBα (data not shown).

Maximal activation of PKB by PDK1/PIF requires PtdIns(3,4,5)P₃ to interact with PDK1. Although PDK1 binds to PtdIns(3,4,5)P₃ with high affinity through its PH domain, this interaction does not influence the ability to activate a PKB mutant lacking the PH domain [18,29]. This is confirmed in FIG. 8 which shows that the rate at which His-PDK1 phosphorylates and activates a mutant form of PKB lacking the PH domain (GST-ΔPH-PKBα) is the same in the presence or absence of PtdIns(3,4,5)P₃ (FIG. 8A). Intriguingly, the addition of GST-PIF* to His-PDK1 halves the rate at which PDK1 activates GST-ΔPH-PKBα in the absence of PtdIns(3,4,5)P₃. However the addition of PtdIns(3,4,5)P₃ results in a 3 to 4-fold enhancement of the rate at which His-PDK1/GST-PIF* can activate GST-ΔPH-PKBα (FIG. 8A). Furthermore, the GST-PIF/PDK1 complex purified from 293 cells also activates GST-ΔPH-PKBα at a ˜4-fold higher rate in the presence of PtdIns(3,4,5)P₃ or PtdIns(3,4)P₂ than in its absence. PtdIns(4,5)P₂ or PtdIns(3)P do not increase the rate at which the GST-PIF/PDK1 complex phosphorylates GST-ΔPH-PKBα (FIG. 8B).

Identification of a PtdIns(3,4,5)P₃ dependent Ser473 kinase activity as PDK1. We fractionated rat brain extracts by batchwise chromatography on Q-Sepharose, followed by gradient elution from Heparin-Sepharose (see methods). PDK1 (assayed as a PtdIns(3,4,5)P₃ dependent enzyme that activates GST-S473D-PKBα) was eluted from Heparin-Sepharose as a broad peak at 0.75M NaCl (FIG. 9A, upper panel). A PtdIns(3,4,5)P₃ dependent PDK2 activity that phosphorylated PKBα at Ser473 was also identified in the Heparin-Sepharose eluate and was present in the tailing half of the PDK1 activity (FIG. 9, lower panel). The peak containing the PDK2 activity (fractions 7 and 8) was pooled, PDK1 removed by immunoprecipitation and the supernatant reassayed for PDK1 and PDK2 activity. Strikingly, the antibody completely removed not only the PDK1 (Thr308 kinase) activity but also the PDK2 (Ser473 kinase) activity (FIG. 9B). This result indicates that a fraction of the PDK1 in the Heparin-Sepharose eluate possesses PDK2 activity.

Discussion

PDK1 has been shown to phosphorylate and activate several of the AGC subfamily of protein kinases in a conserved motif between subdomains VII and VIII of the kinase catalytic domain ([3,13] see introduction). Most of the PDK1 substrates identified to date possess a PDK2 consensus motif ˜160 residues C-terminal to the PDK1 phosphorylation site, apart from PKA whose sequence actually terminates at the second Phe of the PDK2 phosphorylation motif (see FIG. 1E). In PKA it is possible that the C-terminal carboxylate group may mimic the effect of phosphorylation at the PDK2 site, thereby generating a fully active enzyme. Phosphorylation of both the PDK1 and PDK2 sites is required for maximal activation of PKB and p70 S6 kinase and/or stability for PKC isoforms [30]. In this Example, we demonstrate that the kinase domain of PDK1 is capable of interacting specifically and with high affinity with a modified PDK2 phosphorylation consensus motif present at the C-terminus of PRK2, in which the phosphorylated Ser/Thr residue is replaced by Asp (Asp 978) instead of a Ser/Thr residue at the site of phosphorylation. The Asp residue may mimick a phosphorylated PDK2 motif. Mutation of any of the conserved aromatic residues in the PDK2 consensus sequence to Ala or mutation of Asp978 to Ala or Ser completely abolished the interaction of PDK1 with PIF (FIG. 3).

PRK2 itself is likely to be a substrate for PDK1 as it possesses a perfect PDK1 consensus sequence between sub-domain VII and VIII of the kinase domain (TFCGTPEFL (SEQ ID NO:9), where the underlined Thr residue corresponds to Thr816, the putative site of PDK1 phosphorylation). In the case of PRK2, it is possible that the role of the PIF domain is to interact directly with PDK1 ensuring that Thr816 is phosphorylated efficiently by PDK1. As PRK2 [28] (and its close relative PRK1 [31,32]) may be dependent on Rho for activity, it is possible that the interaction between PRK2 and Rho complexed to GTP is also required for the phosphorylation of PRK2 by PDK1 to take place. One scenario is that the C-terminus of PRK2 is exposed upon the formation of a complex between PRK2 and Rho-GTP. This may then enable interaction with and phosphorylation by PDK1 leading to the activation of PRK2. However, PRK2 is known to become proteolysed during apoptosis as a result of cleavage by caspase-3 immediately C-terminal to Aspl 17 and Asp700 [33]. PRK2 is highly susceptible to proteolysis when purified or overexpressed in cells [34]. Therefore, a possibility is that a C-terminal fragment of PRK2 devoid of any catalytic activity, is present in cells that interacts with PDK1. It is also possible that a fragment of PRK2 may be generated by an alternative splicing mechanism or the use of alternative promoters. Future work will need to address whether PDK1 is complexed with PRK2 or fragments of this enzyme in intact cells.

SGK and the p70 S6 kinase are far better substrates for PDK1 if the PDK2 phosphorylation site (Thr389) is changed to an acidic residue, for example Glu [24,25]. Thus PDK1 may be able to interact directly with SGK or the p70S6K when the PDK2 site is phosphorylated enabling PDK1 to phosphorylate these enzymes at the PDK1 site in the activation loop. PDK1 complexed to another protein may be capable of inducing phosphorylation of the PDK2 site of these enzymes. PKCζ which is also activated by PDK1 [21,22], possesses an acidic residue (Glu 579) rather than Ser/Thr in its PDK2 consensus motif (FEGFEY) (SEQ ID NO:10). Furthermore, PKCζ, like PIF, may interact directly with the kinase domain of PDK1.

Activation of PKBα in cells is mediated by phosphorylation of both Thr308 and Ser473. Phosphorylation of either residue only partially activates PKBα and the full activation requires the phosphorylation of Ser473 as well as Thr308 [14]. A catalytically inactive mutant of PKBα becomes phosphorylated at Ser473 in response to insulin, while PKBα phosphorylated at Thr308 alone or in which Thr308 is mutated to Asp does not become phophorylated at Ser473 in vitro upon incubation with MgATP in the presence of PtdIns(3,4,5)P_{3 [}14,17]. These observations indicate that phosphorylation of PKBα at Ser473 is unlikely to be an autophosphorylation event, catalysed by PKBα itself. We have reported previously [14] that Ser473 is phosphorylated in vitro by MAP kinase-activated protein kinase-2 [14], but this enzyme does not mediate the phosphorylation of Ser473 in vivo in response to insulin, IGF1, heat shock or hydrogen peroxide [35]. Furthermore, the phosphorylation of PKBα at Ser473 by MAPKAPK-2 in vitro is not dependent upon the presence of PtdIns(3,4,5)P_{3 [}14]. It has also been claimed that the integrin-linked kinase (ILK) is capable of phosphorylating Ser473 of PKBα in vitro [36]. We have not been able to demonstrate that ILK phosphorylates Ser473 of PKBα, and it is also uncertain as to whether ILK is actually a protein kinase as it lacks certain motifs found in every other protein kinase. It has also been claimed that mTOR can phosphorylate the PDK2 site of p70S6 kinase [37]; however, we were unable to reproduce this finding and furthermore were unable to phosphorylate PKBα at Ser473 with mTOR.

Since the insulin/IGF1-induced phosphorylation of Ser473, like that of Thr308, is prevented by inhibitors of PI 3-kinase, it is likely that phosphorylation of Ser473 is also catalysed by a PtdIns(3,4,5)P₃ dependent protein kinase. In this study we have made the surprising observation that the interaction of PIF with the kinase domain of PDK1 converts it from an enzyme that only phosphorylates PKBα at Thr308 to a form that phosphorylates both Thr308 and Ser473 in a PtdIns(3,4,5)P₃ and PtdIns(3,4,)P₂ dependent manner. The requirement for PtdIns(3,4,5)P₃ or PtdIns(3,4,)P₂ is extremely specific, because only the D-enantiomers of PtdIns(3,4,5)P₃ are effective, and many other PtdIns phospholipids including PtdIns(4,5)P₂ are ineffective. The residues surrounding Thr308 and Ser473 are distinct, the only common feature being an aromatic residue at the residue immediately C-terminal to the phosphorylation site. This suggests that PDK1 possesses two distinct specificities when complexed to PIF.

If a mutant of PKBα lacking the PH domain (which cannot interact with PtdIns(3,4,5)P₃) is expressed in cells, it nevertheless undergoes a wortmannin-sensitive activation and phosphorylation at Thr308 and Ser473 [38]. This implies that PDK1 is activated by PtdIns(3,4,5)P₃ in vivo. However, PDK1 isolated from either control or insulin stimulated cells possessed similar activity towards GST-ΔPH-PKBα [18,25]. Furthermore, in vitro experiments demonstrated that despite binding PtdIns(3,4,5)P₃/PtdIns(3,4)P₂ through its PH domain [20, 39], these 3-phosphoinositides have no influence on the rate at which PDK1 phosphorylates ΔPH-PKBα, p70S6 kinase or SGK substrates which do not interact with 3-phosphoinositides [3, 43, 44]. Importantly, the interaction with PIF confers on PDK1 the ability to be activated by PtdIns(3,4,5)P₃/PtdIns(3,4)P₂ in vitro (FIG. 8). This does not happen if the PH domain of PDK1 is deleted (data not shown). Thus PDK1 complexed to PIF activates GST-ΔPH-PKBα at a ˜3 to 4-fold higher rate in the presence of PtdIns(3,4,5)P₃ than in its absence. Our data suggest that the interaction of PDK1 with PIF leads to a ˜2-fold inhibition of the catalytic activity of PDK1 which is relieved and further activated in the presence of PtdIns(3,4,5)P₃.

The results presented in this paper raise the strong possibility that PDK1 itself may be the enzyme responsible for mediating the phosphorylation of PKBα at both Thr308 and Ser473. It is possible that PRK2 (or a proteolytic fragment of PRK2) or a related peptide/protein(s) may interact with the kinase domain of PDK1, converting it into an enzyme that can phosphorylate both Ser473 and Thr308 of PKBα, as well as permitting the direct activation of PDK1 by PtdIns(3,4,5)P₃. Consistent with this hypothesis we have identified a PtdIns(3,4,5)P₃ dependent Ser473 kinase activity that is immunoprecipitated with a PDK1-specific antibody (FIG. 9). This form of PDK1 may be complexed to PRK2 or its C-terminal fragment or to another peptide/protein with similar properties. The overexpression of PDK1 in cells has been shown to potentiate the phosphorylation of PKBα at Thr308 only [18], which might be explained if the putative PDK1 regulatory subunits are present in limiting amounts.

In summary, the present work provides the first evidence that PDK1 and PDK2 may be the same enzyme and that its specificity for its two different phosphorylation substrate sites may be modulated by the interaction with one or more proteins. Our results also suggest that when PDK1 interacts with the PH domain of PDK1 with PtdIns (3,4,5)P₃/PtdIns(3,4)P₂.

Materials and Methods

Materials. Peptides were synthesised by Dr G. Blomberg (University of Bristol, U.K.). Protein G-Sepharose, glutathione Sepharose and CHX-Sepharose were purchased from Pharmacia (Milton Keynes, UK), alkylated trypsin from Promega (Southampton, UK), tissue culture reagents, microcystin-LR, Life Technologies Inc. (Paisley, UK), the pCR 2.1-TOPO cloning vector from Invitrogen (Leek, Netherlands), sensor Chips CM5 and SA were from BiaCore AB and biotinylated reagent was from Pierce. The yeast two hybrid human skeletal muscle (in pGAD10 vector) and the pAS2-1 vector were purchased from Clontech (Basingstoke, UK). Expression constructs for GST-PKBα [17], GST-PDK1, Myc-PDK1 and other mutants of PDK1 [18], GST-90 RSK1 [39], GST-MSK1 [39] and GST-p70 S6 kinase lacking the C-terminal 104 residues [24] were generated as described previously. Synthesis of 1-[(1-O-Stearoyl-2-O-arachidonoyl-sn-glycer-3-yl)-phosphoryl]-D-myo-inositol 3,4,5-trisphosphate [PtdIns(3,4,5)P₃] and its stereoisomers is described in [40], all lipids were obtained from sources described previously [17]. All the experiments using 3-phosphoinositides were carried out in the presence of phospholipids containing 100 μM Phosphatidylcholine and 100 μM Phosphatidylserine.

Antibodies. The PDK1 whole protein antibody was raised in sheep against GST-PDK1 expressed and purified from 293 cells. The antibody was affinity-purified on CH-Sepharose columns to which His-PDK1 had been covalently coupled. The peptide PDK1 antibody was raised in sheep against the peptide RQRYQSHPDAAVQ (SEQ ID NO:11) (corresponding to residues 544 to 556 of PDK1) and affinity-purified on CH-Sepharose columns to which this peptide was coupled covalently. The phospho-specific antibody recognising PKB phosphorylated at Ser 473 was raised against the peptide PHFPQFSYSAS (SEQ ID NO:8) in which the underlined serine is phosphorylated (corresponding to residues 467 to 477 of PKBα) and affinity purified on CH-Sephaose covalently coupled to the phosphorylated peptides. The antibodies were then passed through a column coupled to the non-phosphorylated peptide and the antibodies that did not bind to this column were selected. The above antibodies are available commercially from UBI (Lake Placid, USA). A monoclonal antibody recognising the Myc epitope was purchased from Boehringer Mannheim (Lewes, UK).

Buffer solutions. Buffer A—50 mM Tris-HCl pH 7.5, 1 mM EGTA, 1 mM EDTA, 1% (by mass) Triton-X 100, 1 mM sodium orthovanadate, 50 mM sodium fluoride, 5 mM sodium pyrophosphate, 0.27 M sucrose, 1 μM microcystin-LR, 0.1% (by vol) β-mercaptoethanol and ‘complete’ proteinase inhibitor cocktail (one tablet per 50 ml; Boehringer Mannheim, Lewes, UK). Buffer B—50 mM Tris/HCl pH 7.5, 0.1 mM EGTA, 10 mM β-mercaptoethanol. Buffer C—50 mM Tris/HCl pH 7.5, 1 mM EDTA, 1 mM EGTA, 0.27M sucrose and 0.1% (by vol) β-mercaptoethanol.

General methods. E. coli cells (typically DH5) were grown at 37° C. in Luria-Bertani medium containing (when needed) 50 μg/ml ampicillin for plasmid selection. Restriction enzyme digests, DNA ligations and other recombinant DNA procedures were performed as using standard protocols. Transformation of bacterial cells was achieved using electroporation. Site-directed mutagenesis was performed using the QuikChange Kit (Strategene) following instructions provided by the manufacturer. All DNA constructs were verified by automatic DNA sequencing using an automated DNA Sequencer (Model 373; Applied Biosystems). DNA constructs were used in this study were purified from bacteria using Qiagen plasmid Mega kit according to the manufacturer's protocol. 293 cells were cultured to confluence on 10 cm diameter dishes in Dulbecco's Modified Eagle's Medium containing 10% (by vol) foetal bovine serum. Ptd/Cho and Ptd/Ser phospholipid vesicles containing the indicated phosphoinositide lipids were prepared as described previously [17].

Yeast Two Hybrid Screen.

Myc-tagged human PDK1, kinase domain of PDK1 (residues 1-404) and the PH domain of PDK1 (428 to 556) were subcloned into the EcoRI/SalI of the yeast pAS2-1 vector. A yeast two hybrid screen was carried out using a human skeletal muscle library subcloned into the pGAD10 vector (Clontech) transformed into the yeast strain Y166. 4×10⁶ yeast cells were transformed with pGAD10 library constructs and PDK1 constructs in the pAS2-1 vector. In order to select positive colonies these yeast were plated onto synthetic complete (SC) media lacking tryptophan, leucine, histidine and uracil (SC-Trp-Leu-His-Ura), containing 25 mM 3-amino-1,2,4-triazole (3-AT) (Sigma). In this system interacting clones from the library are able to grow in medium lacking histidine and uracil and express β-galactosidase which was detected using the Clontech protocol. pGAD10 plasmids from interacting clones were isolated transferred to E. coli, and sequenced. One positive clone termed by us TH110, contained a 2.4 kbp fragment which possessed in the 5′ region a 25 nucleotide sequence from a rRNA, followed by the 77 C-terminal amino acids of PRK2 (termed PIF) in frame with the GAL4 protein. In order to eliminate the 25 nucleotide rRNA sequence from this construct, a PCR reaction was carried out using, as a template, the TH110 clone and the oligonucleotides 5′-CGGGATCCGAGGATGTAAAAAAGCACCC-3′ (SEQ ID NO:12) (incorporating a BamH1 site which is underlined) and an oligonucleotide corresponding to the polylinker region at the 3′ end of the pGAD10 vector. The resulting fragment contains an internal BamH1 site and the PCR product was digested with BamH1 to obtain a 1.2 kb fragment encoding PIF which was ligated into the pGAD10 plasmid. This construct was used in the experiments in FIG. 1 to demonstrate that PIF interacts with the kinase domain of PDK1 in the yeast two hybrid system.

Preparation of mammalian GST-PIF expression constructs. A ligation was set up to generate a GST-PIF expression construct by subcloning the PIF cDNA from the pGAD10 vector as the BamH1 fragment into the BamH1 site of the pEBG2T [41] expression vector.

Expression of His-PDK1 in insect cells. The cDNA for Myc-PDK1 was subcloned from pEBG2T vector [41] as a BglHI/KpnI fragment into pFASTBAC HTb vector and this vector was used to generate recombinant baculovirus using the Bac-to-Bac system (Life Technologies, Paisley, UK). The resulting virus encodes Myc-PDK1 with an N-terminal hexahistidine sequence, and was used to infect Sf21 cells (1.5×10⁶/ml) at a multiplicity of infection of 5. The infected cells were harvested 72 h post-infection and the His-PDK1 purified by Ni²⁺/NTA-agarose chromatography as described previously for His-PKBβ [42], and dialysed into Buffer B containing 0.27 M sucrose, 0.03% (by vol) Brij-35, 1 mM benzamidine and 0.2 mM phenylmethylsulphonyl fluoride. The PDK1 (4 mg/ml) was snap frozen in aliquots and stored at −80° C. Purified His-PDK1 was recovered with a yield of 4 mg/litre of infected Sf21 cells and was >90% homogeneous as judged by polyacrylamide gel electrophoresis followed by Coomassie Blue staining.

Expression of GST-fusion proteins in 293 cells. Up to twenty 10 cm diameter dishes of 293 cells were cultured and each dish transfected with 20 μg of pEBG2T DNA encoding the desired GST-fusion protein using a modified calcium phosphate method [14]. In experiments described in FIG. 1 in which Myc-PDK1 constructs (pCMV5 vector) were coexpressed with GST-PIF (pEBG2T vector) or GST alone (empty pEBG2T vector) 10 μg of each construct was used. 36 h after transfection the cells were lysed in 1 ml of ice-cold Buffer A without any serum starvation. The lysates were pooled, centrifuged at 4° C. for 10 min at 13,000×g and the supernatant incubated for 60 min on a rotating platform with 1 ml of glutathione-Sepharose (for a 20 dishes experiment) previously equilibrated in Buffer A. The suspension was centrifuged for 1 min at 3000×g, the beads washed three times with 10 ml of Buffer A containing 0.5 M NaCl, and then a further ten times with 10 ml of Buffer B containing 0.27 M sucrose. GST-PIF was eluted from the resin at ambient temperature with three volumes (equivalent to the GST-Sepharose volume) of Buffer B containing 20 mM glutathione and 0.27 M Sucrose. The combined eluates were divided into aliquots, snap frozen in liquid nitrogen, and stored at −80° C.

Measurement of PKBα activation after incubation with GST-PIF. This assay was carried out in 2 stages, as described previously [17]; briefly, in the first, GST-PKBα was incubated with GST-PIF (or other indicated PDK1 proteins in the presence or absence of peptides) in the presence of MgATP and phospholipid vesicles. In the second stage, the solution was made 0.5% (by vol) in Triton X100 (which completely inhibits phosphorylation of GST-PKBα without affecting GST-PKBα activity [17]) together with Mg[γ³²P]ATP, and the specific PKBα substrate peptide substrate RPRTAAF (SEQ ID NO:13) [43].

In Stage 1, an 15 μl reaction mixture was set up containing 66.5 mM Tris/HCl pH 7.5, 0.13 mM EGTA, 0.13% (by vol) 2-mercaptoethanol, 3.3 μM PKI, 1.3 μM microcystin-LR, 13.3 mM Mg(Ac)₂, 133 μM unlabelled ATP, 0.4 μM GST-PKBA, 133 μM PtdSer, 133 μM PtdCho in the presence or absence of 13.3 μM PtdIns(3,4,5)P₃. The assay was initiated by the addition of 5 μl of 0.2 mg/ml GST-PIF (or other indicated proteins or peptides), after incubation for 30 min at 30° C., stage 2 of the assay was initiated by the addition of 30 μl of a mixture made up of Buffer B containing 2.5 μM PKI, 1 μM microcystin-LR, 10 mM Mg(Ac)₂, 100 μM [γ³²P]ATP (200-400 cpm/pmol), 100 μM of the peptide RPRTAAF (SEQ ID NO:13) and 1.25% (by vol) Triton X-100. After 10 min at 30° C., the reactions were terminated by spotting the reaction mixture on to P81 phosphocellulose paper. The papers were washed in 75 mM phosphoric acid and analysed as described previously [44]. A control reaction in which GST-PKBα was omitted was taken as the blank and was always less than 5% of the activity measured in the presence of GST-PKBα. The basal GST-PKBα activity is the activity measured in the absence of PDK1. 1 Unit of GST-PKBα activity was that amount of enzyme required to catalyse the phosphorylation of 1 nmol of the peptide RPRTAAF (SEQ ID NO:13) in 1 min. The assays were linear with time up to a final concentration of 3 U/ml of PDK1 activity in the assay.

Phosphorylation of GST-PKBα by incubation with GST-PIF. In experiments in which the phosphorylation of PKBα was determined using the phospho-specific Ser473 PKBα antibodies, the incubations were identical to the Stage 1 described above except that the reactions were terminated by the addition of 1% (by mass) SDS and immunoblotting is carried out as described below. In experiments in which the sites of phosphorylation on PKBα are determined the incubations were identical to the Stage 1 described above except [γ³²P] ATP (1000-2000 cpm/pmol) was used instead of unlabelled ATP. The reactions were stopped by the addition of SDS and 2-mercaptoethanol to final concentrations of 1% (by mass) and 1% (by vol) respectively, and heated for 5 min at 95° C. After cooling to ambient temperature, 4-vinylpyridine was added to a concentration of 2.5% (by vol) and the sample left on a shaking platform for 1 h at 30° C. to alkylate cysteine residues. The sample was then electrophoresed on a 7.5% SDS polyacrylamide gel, the ³²P-labelled GST-PKBα was eluted from the gel and digested with alkylated trypsin as described previously [14, 17].

Immunoblotting for PKBα phosphorylated at Ser473. Reaction mixtures containing GST-PKBα (0.2 μg) were subjected to SDS/polyacylamide gel electrophoresis and subsequently transferred to nitrocellulose and immunoblotted using the phospho-specific antibody recognizing PKBα phosphorylated on Ser 473 at a concentration of antibody of 0.1 mg/ml in 50 mM Tris pH7.5, 0.15M NaCl, 0.5% (by vol) Tween and 10% (by mass) skimmed milk. Detection of phosphorylated PKBα was performed using the enhanced chemiluminescence reagent (Amersham).

BiaCore measurements. GST-PIF was coupled through amine groups to the Sensor Chip CM5 according to the manufacturer's instructions. His-PDK1 (0-2.0 μM) was injected over this surface and steady state binding determined. The 24 residue peptide overlapping Region B was biotinylated through its C-terminal Cys and bound to Avidin coated Sensor Chip SA. His-PDK1 (0.5 μM) was mixed with region B peptide (0-3 μM) and the PDK1/peptide mixtures injected over the immobilised peptides. The decrease in steady state binding was used to determine the Kd of interaction between PDK1 and the peptide, according to the Cheng-Prescott relationship. The measure of response in our experiments is termed RU; 1000 RU=1 ng/mm² of protein bound to the surface.

Partial Purification of PDK2 from Rat Brain.

Ten rats were killed with CO₂, the brains rapidly extracted and homogenised in 50 ml of ice-cold Buffer A. After centrifugation for 30 min at 25000×g, the supernatant was filtered through a 0.44 micron filter and applied to a 5 ml HiTrap Q-Sepharose column equilibrated in Buffer C. The column was washed with 100 ml of Buffer C containing 0.1M NaCl, and then eluted with 30 ml of Buffer C containing 0.3 M NaCl. The eluate was diluted to 0.2M NaCl in Buffer C, and applied directly on to a 1 ml heparin-Sepharose column (HiTrap) equilibrated in Buffer C containing 0.2 M NaCl. The column was developed with a 20 ml linear salt gradient to 2.0M NaCl at a flow rate of 1 ml/min and fractions of 1 ml were collected.

### REFERENCES

1. Vanhaesbroeck B., Leevers S. J., Panayotoy G and Waterfield M. D: Phosphoinositide 3 kinases: a conserved family of signal transducers. Trends Biochem. Sci. 1997, 22: 276-272.
2. Shepherd P. R., Withers D. J and Siddle K: Phosphoinositide 3-kinase: the key switch in insulin signalling. Biochem. J. 1988, 333: 471-479.
3. Alessi, D. R. and Downes, C. P. The role of PI 3-kinase in insulin action. Biochem. Biophys. Acta 1998, 1436: 151-164.
4. Woscholski R and Parker P J Inositol lipid 5-phosphatases—traffic signals and signal traffic. Trends Biochem. Sci. 1997 22: 427-431.
5. Myers M. P., Pass I., Batty I. H., VanderKaay J., Stolarov J. P., Hemmings B. A., Wigler M. H., Downes C. P. and Tonks N. K. The lipid phosphatase activity of PTEN is critical for its tumor suppressor function. Proc. Natl. Acad. Sci. 1998, 95: 13513-13518.
6. Maehama T, Dixon J. E. The tumor suppressor, PTEN/MMAC1, dephosphorylates the lipid second messenger, phosphatidylinositol 3,4,5-trisphosphate. J. Biol. Chem. 1998, 273: 13375-13378
7. Kohn A D, Kovacina K S. and Roth R A: Insulin stimulates the kinase-activity of rac-pk, a pleckstrin-homology domain-containing ser/thr kinase. EMBO J. 1995, 14: 4288-4295.
8. Cross D. A. E., Alessi D. R., Cohen P., Andjelkovic M., and Hemmings B. A. Inhibition of glycogen-synthase kinase-3 by insulin is mediated by protein-kinase-b. Nature 1995, 378: 785-789.
9 Deprez J., Vertommen D., Alessi D. R., Hue L. and Rider M. H. Phosphorylation and activation of heart 6-phosphofructo-2-kinase by protein kinase-B and other protein kinases of the insulin signalling cascades. J. Biol. Chem. 1997, 272: 17269-17275.
10. Barthel A., Kohn A. D., Luo Y. N. and Roth R. A A constitutively active version of the Ser/Thr kinase Akt induces production of the ob gene product, leptin, in 3T3-L1 adipocytes. Endocrinology 1997, 138: 3559-3562.
11. Liao J. F., Barthel A., Nakatani K and Roth R. A. Activation of protein kinase B/Akt is sufficient to repress the glucocorticoid and cAMP induction of phosphoenolpyruvate carboxykinase gene. J. Biol. Chem. 1998, 273: 27320-27324.
12. Welsh G. I., Miller C. M., Loughlin A. J., Price N. T and Proud C. G. Regulation of eukaryotic initiation factor eIF2B: glycogen synthase kinase-3 phosphorylates a conserved serine which undergoes dephosphorylation in response to insulin. FEBS LETT. 1998, 421: 125-130.
13. Alessi D. R. and Cohen P. Mechanism of activation and function of protein kinase B. Curr. Opin. Genetics. Develop. 1998, 8: 55-62.
14 Alessi D. R., Andjelkovic M., Caudwell F. B., Cron P., Morrice N., Cohen P. and Hemmings B. Mechanism of activation of protein kinase B by insulin and IGF1. EMBO J.1996 15: 6541-6551.
15. Pearson R. B., Dennis P. B., Han J. W., Williamson N. A., Kozma S. C., Wettenhall, R. E. H. and Thomas G (1995) The principal target of rapamycin-induced p70s6k inactivation is a novel phosphorylation site within a conserved hydrophobic domain. EMBO J. 14: 5278-5287.
16. Mellor H and Parker P. J. The extended protein kinase C superfamily. Biochem. J. 1998, 332: 281-292.
17. Alessi D. R., James S. R., Downes C. P., Holmes A. B., Gaffney P. R. J., Reese C. B. and Cohen P. Characterization of a 3-phosphoinositide-dependent protein kinase which phosphorylates and activates protein kinase B. Curr. Biol. 1997, 7: 261-269.
18. Alessi D. R., Deak M., Casamayor A., Caudwell F. B., Morrice N., Norman, D. G., Gaffney P., Reese C. B., MacDougall C. N., Harbison D., Ashworth A. and Bownes M. 3-phosphoinositide-dependent protein kinase-1 (PDK1): structural and functional homology with the Drosophila DSTPK61 kinase. Curr. Biol. 1997, 7: 776-789.
19. Stokoe D., Stephens L. R., Copeland T., Gaffney P. R. J., Reese C. B., Painter G. F. Dual role of phosphatidyinositiol-3,4,5-trisphosphate in the activation of protein kinase B. Science, 1997, 277: 567-570.
20. Stephens L., Anderson K., Stokoe D., Erdjument-Bromage H., Painter G. F., Holmes A. B., Gaffney P. R. J., Reese C. B., McCormick F., Temps P., Coadwell J. and Hawkins P. T. Protein kinase B kinases that mediate phosphatidylinositol 3,4,5-trisphosphate-dependent activation of protein kinase B. Science 1998, 279: 710-714.
21. LeGood J. A., Ziegler W. H., Parekh D. B., Alessi D. R., Cohen P. and Parker P. J. Protein kinase C isotypes controlled by phosphoinositide 3-kinase through the protein kinase PDK1. Science 1998, 281: 2042-2045.
22. Chou M. M., Hou W. M., Johnson J., Graham L. K., Lee M. H., Chen C. S., Newton A. C., Schaffhausen B. S. and Toker A. Regulation of protein kinase C zeta by PI 3-kinase and PDK1. Curr. Biol. 1998, 8: 1069-1077.
23. Dutil, E. M., Toker, A. and Newton, A. C. Regulation of conventional protein kinase C isozymes by phosphoinositide-dependent kinase 1 (PDK-1). Curr. Biol. 1988, 8:1366-1375.
24. Alessi D. R., Kozlowski M. T., Weng Q. P., Morrice N. and Avruch, J. 3-phosphoinositide dependent protein kinase 1 (PDK1) phosphorylates and activates the p70S6 kinase in vivo and in vitro. Curr. Biol. 1998, 8: 69-81.
25. Pullen N., Dennis P. B., Andjelkovic M., Dufner A., Kozma S. C., Hemmings B. A. and Thomas G. Phosphorylation and activation of p70(s6k) by PDK1. Science 1998, 279: 707-710.
26. Cheng X. D., Ma Y. L., Moore M., Hemmings B. A., Taylor S. S. Phosphorylation and activation of cAMP-dependent protein kinase by phosphoinositide-dependent protein kinase. Proc. Natl. Acad. Sci. USA 95: 9849-9854.
27. Palmer, R. H., Ridden, J. and Parker, P. J. Cloning and expression patterns of 2 members of a novel protein-kinase C-related kinase family. Eur. J. Biochem. 1995, 227: 344-351.
28. Vincent S. and Settleman J. The PRK2 kinase is a potential effector target of both Rho and Rac GTPases and regulates actin cytoskeletal organization. Mol. Cell. Biol. 1997, 17: 2247-2256.
29. Currie, R. A., Walker, K. S., Gray, A., Deak, M., Casamayor, A., Downes, C. P., Cohen, P., Alessi, D. R. and Lucocq, J. The role of phosphatidylinositol 3,4,5-trisphosphate in regulating the activity and localisation of 3-phosphoinositide-dependent protein kinase-1. Biochem. J. in the press.
30. Bornancin F and Parker P. J. Phosphorylation of threonine 638 critically controls the dephosphorylation and inactivation of protein kinase C alpha. Curr. Biol. 1996,6: 1114-1123
31. Amano, M., Mukai, H., Ono, Y., Chihara, K., Matsui, T., Hamajima, Y., Okawa, k., Iwamatsu, A. and Kaibuchi, K. Identification of a putative target for Rho as the Serine-threonine kinase N. Science 1996, 271: 648-650.
32. Vincent, S. and Settleman, J. The PRK2 kinase is a potential effector target of both Rho and Rac GTPases and regulates actin cytoskeletal organization. Mol Cell. Biol. 1997, 17:, 2247-2256.
33. Cryns V. L., Byun Y., Rana A., Mellor H., Lustig K. D., Ghanem. L., Parker P. J., Kirschner M. W., Yuan J. Y. Specific proteolysis of the kinase protein kinase C-related kinase 2 by caspase-3 during apoptosis—Identification by a novel, small pool expression cloning strategy. J. Biol. Chem. 1997, 272: 29449-29453.
34 Yu, W., Liu, J., Morrice, N. A. and Wettenhall, R. E. H. Isolation and characterisation of a structural homologue of human PRK2 from rat liver. J. Biol. Chem. (1997) 272: 10030-10034
35. Shaw, M, Cohen, P and Alessi, D, R. The activation of protein kinase B by H2O2 or heat shock is mediated by phosphoinositide 3-kinase and not by mitogen-activated protein activated protein kinase-2. Biochem. J. 1998, 336: 241-246.
36. Burnett P. E., Barrow R. K., Cohen N. A., Snyder S. H., Sabatini D. M. RAFT1 phosphorylation of the translational regulators p70 S6 kinase and 4E-BP1. Proc. Natl. Acad. Sci. 1998, 95: 1432-1437.
37. Delcommenne M., Tan C., Gray V., Rue L., Woodgett J. and Dedhar S. Phosphoinositide-3-OH kinase-dependent regulation of glycogen synthase kinase 3 and protein kinase B/AKT by the integrin-linked kinase. Proc. Natl. Acad. Sci. 1998, 95: 11211-11216
38. Andjelkovic M, Alessi D. R., Meier R., Fernandez A., Lamb N. J. C., Frech M., Cron P., Cohen P., Lucocq J. M. and Hemming B. A: Role of translocation in the activation and function of protein kinase B. J. Biol. Chem. 1997, 272: 31515-31324.
39. Deak M., Clifton A. D., Lucocq J. M. and Alessi D. R. Mitogen- and stress-activated protein kinase-1 (MSK1) is directly activated by MAPK and SAPK2/p38, and may mediate activation of CREB. EMBO J. 1998 17: 4426-4441.
40. Gaffney, P. R. J. and Reese C. B. Synthesis of 1-[(1-O-Stearoyl-2-O-arachidonoyl-sn-glycer-3-yl)-phosphoryl]-D-myo-inositol 3,4,5-trisphosphate [PtdIns(3,4,5)P3] and its stereoisomers. Bioorg. Med. Chem. Lett., 1997, 7: 3171-3176.
41. Sanchez, I., Hughes, R. T., Mayer, B. J., Yee, K., Woodgett, J. R., Avruch, J., Kyriakis, J. M. and Zon, L. I. Role of SAPK/ERK kinase-1 in the stress-activated pathway regulating transcription factor c-Jun. Nature, 1994, 372: 794-798.
42. Walker K. S., Deak M., Paterson A., Hudson K., Cohen P., Alessi. D. R. Activation of protein kinase B beta and gamma isoforms by insulin in vivo and by 3-phosphoinositide-dependent protein kinase-1 in vitro: comparison with protein kinase B alpha. Biochem. J. 1998, 331: 299-308.
43. Alessi D. R., Caudwell F. B., Andjelkovic M., Hemmings B. A. and Cohen P: Molecular basis for the substrate specificity of protein kinase B; comparison with MAPKAP kinase-1 and p70S6 kinase. FEBS Lett. 1996, 399: 333-338.
44. Alessi, D. R., Cohen, P., Leevers, S., Cowley, S. and Marshall, C. J. (1995) Assay and expression of Mitogen-Activated protein kinase, MAP kinase kinase and Raf. Methods Enzymol. 255: 279-290.
45. Kobayashi T, Cohen P (1999) Activation of SGK by PDK1. Biochem J In the press.
46. Casamayor A., Torrance, P. D., Kobayashi, T., Thorner, J. and Alessi, D. R. (1999) Functional counterparts of mammalian protein kinases PDK1 and SGK in budding yeast. Curr Biol 9, 186-197.
47. Gaffney, P. R. J. & Reese, C. B. (1997) Synthesis of 1-[(1-O-Stearoyl-2-O-arachidonyl-sn-glycer-3-yl)-phosphoryl]-D-myo-inositol 3,4,5-triphosphate [PtdIns(3,4,5)P₃] and its stereoisomers. Bioorg. Med. Chem. Lett 7, 3171-3176.

1. A method of altering the substrate specificity of a phosphoinositide-dependent protein kinase 1 (PDK1) wherein the PDK1 is exposed to an interacting polypeptide which comprises an amino acid sequence corresponding to SEQ ID NO:25.

2. A preparation comprising PDK1 and an interacting polypeptide which comprises an amino acid sequence corresponding to SEQ ID NO: 25 characterised in that said preparation is substantially free of polypeptides with which PDK1 is present or associated in a cell in which it is naturally found other than said interacting polypeptide or a substrate for PDK1.

3. A method of phosphorylating a residue corresponding to the underlined residue in a substrate polypeptide with an amino acid sequence corresponding to the consensus sequence Phe/Tyr-Xaa-Xaa-Phe/Tyr-Ser/Thr-Phe/Tyr (SEQ ID NO:30) wherein the substrate polypeptide is exposed to (1) a preparation according to claim 2 or (2) a PDK1 having altered substrate specificity wherein said PDK1 having altered substrate specificity is provided by exposing the PDK1 to the interacting polypeptide.

4. A method of identifying a compound that modulates the activity of PDK1 comprising:
exposing said PDK1 to said compound in the presence of a polypeptide comprising the amino acid sequence corresponding to SEQ ID NO:25.

5. A method according to claim 4 further comprising:
measuring the activity of said PDK1 in the presence of more than one concentration of the compound wherein said PDK1 is or has been exposed to a polypeptide which comprises the amino acid sequence corresponding to SEQ ID NO:25.

6. A method according to claim 4 wherein said compound is capable of modulating the interaction between a polypeptide which comprises the amino acid sequence corresponding to SEQ ID NO:25 and PDK1.

7. A method of identifying a compound that is capable of mimicking the effect of a 3-phosphoinositide, for example PtdIns(3,4,5)P₃ or PtdIns(3,4)P₂, on the PDK1 or PDK2 activity of (1) a PDK1 which has altered substrate specificity or (2) a preparation according to claim 2, the method comprising determining whether said compound activates said PDK1 or preparation according to claim 2 so that it can phosphorylate a suitable substrate, the activation by said compound being in the absence of a 3-phosphoinositide.

8. A polypeptide which comprises the amino acid sequence corresponding to SEQ ID NO: 30 wherein said polypeptide is not full-length PRK2, PRK1 or PKCζ and wherein Zaa is a negatively charged amino acid that is not phosphoserine or phosphothreonine.

9. A method of identifying a compound that is capable of altering the substrate specificity of PDK1 wherein the ability of said PDK1 to phosphorylate a residue of a polypeptide according to claim 8 corresponding to the underlined residue in the polypeptide Phe/Tyr-Xaa-Xaa-Phe/Tyr-Ser/Thr-Phe/Tyr (SEQ ID NO:30) is measured, and is increased in the presence of said compound.

10. A method of altering the substrate specificity of phosphoinositide-dependent protein kinase 1 (PDK1) comprising:
identifying a compound according to claim 9 and
exposing said PDK1 to the compound.

11. A protein kinase derivable from mammalian brain wherein said protein kinase is capable of phosphorylating a residue of a polypeptide according to claim 8 corresponding to the underlined residue in a polypeptide with an amino acid sequence corresponding to the consensus sequence Phe/Tyr-Xaa-Xaa-Phe/Tyr-Ser/Thr-Phe/Tyr (SEQ ID NO:30), for example Ser473 of PKBα in the presence of PtdIns(3,4,5)P₃, wherein the said protein kinase is eluted from Heparin-Sepharose by at least 0.75M NaCl at pH 7.5 and is capable of binding to an antibody reactive with PDK1.

12. A polypeptide according to claim 8 consisting essentially of residues 51 to 404 of PDK1 or a fusion of a polypeptide consisting essentially of residues 51 to 404 of PDK1.

13. A polynucleotide encoding a polypeptide as defined in claim 12.

14. A recombinant polynucleotide suitable for expressing a polypeptide as defined in claim 13.

15. A host cell comprising a polynucleotide as defined in claim 14.

16. A cell containing a recombinant nucleic acid suitable for expressing PDK1 and a recombinant nucleic acid suitable for expressing a polypeptide according to claim 8.

17. A medicament for the treatment of a patient in need of modulation of the insulin signalling pathway and/or PDK1, PDK2 or PRK2 signalling comprising:
a compound identified according to claim 4 and
an effective carrier.

18. A kit of parts for identifying a compound according to claim 4 wherein the kit comprises PDK1 and a polypeptide comprising the amino acid sequence corresponding to SEQ ID NO:25.

19. A method of making a preparation that comprises PDK1 and a polypeptide wherein substantially pure PDK1 is mixed with a substantially pure polypeptide according to claim 8.

20. A method according to claim 4 wherein said PDK1 has at least 65% amino acid identity with SEQ ID NO:31 and retains the enzyme activity of PDK1.

21. A method according to claim 20 wherein said polypeptide has from 10 to 100 amino acid residues.
